(19) United States
(12) Patent Application Publication
(10) Pub. No.: US 2007/0270417 A1
(43) Pub. Date: Nov. 22, 2007

(54) Antibacterial Agents

(75) Inventors: William Henry Miller, Collegeville, PA (US); Meagan B. Rouse, Collegeville, PA (US); Mark Andrew Seefeld, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, GREENFORD, MIDDLESEX (GB)

(21) Appl. No.: 11/570,777

(22) PCT Filed: Jul. 21, 2005
(86) PCT No.: PCT/US05/25843

§ 371 Date: Dec. 18, 2006

Related U.S. Application Data
(60) Provisional application No. 60/590,174, filed on Jul. 22, 2004.

(51) Int. Cl. A61K 31/542; A61P 31/02; A61P 31/04; C07D 513/04

(52) U.S. Cl. 514/224.2; 544/48

(57) ABSTRACT

Naphthalene, quinoline, quinoxaline and naphthyridine derivatives useful in the treatment bacterial infections in mammals, particularly humans, are disclosed herein.

### FIELD OF THE INVENTION

This invention relates to novel compounds, compositions containing them, processes for preparing them and their use as antibacterials.

### BACKGROUND OF THE INVENTION

The emergence of pathogens resistant to known antibiotic therapy is becoming a serious global healthcare problem (Chu, et al., (1996) J. Med. Chem., 39: 3853-3874). Thus, there is a need to discover new broad spectrum antibiotics useful in combating multidrug-resistant organisms. Importantly, it has now been discovered that certain compounds have antibacterial activity, and, therefore, may be useful for the treatment of bacterial infections in mammals, particularly in humans.

### SUMMARY OF THE INVENTION

This invention comprises compounds of the formula (I), as described hereinafter, which are useful in the treatment of bacterial infections. This invention is also a pharmaceutical composition comprising a compound according to formula (I) and a pharmaceutically acceptable carrier. This invention is also processes for the preparation of compounds of formula (I). This invention is also a method of treating bacterial infections in mammals, particularly in humans.

### DETAILED DESCRIPTION OF THE INVENTION

This invention provides a compound of formula (I) or a pharmaceutically acceptable salt, solvate or derivative thereof:

In some aspects, this invention describes a compound of formula (I)

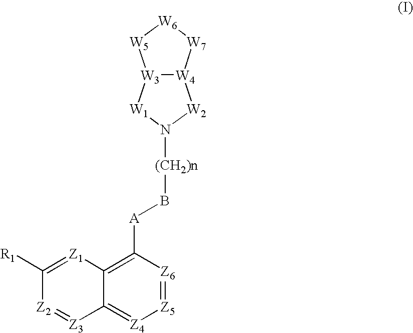

wherein:

Z₁, Z₃, and Z₄ are independently N or CR^{1a};

Z₂, Z₅, and Z₆ are each CR^{1a};

R₁ and R^{1a} are independently at each occurrence hydrogen; cyano; halogen; hydroxy; (C₁₋₆)alkoxy unsubstituted or substituted by (C₁₋₆)alkoxy, hydroxy, amino, piperidyl, guanidino or amidino any of which is unsubstitued or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C-₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C-₁₋₆)alkyl, acyl or (C₁₋₆)alkylsulphonyl groups; or R₁ and R^{1a} of Z₂ together form ethylenedioxy;
AB is NR^{1b}(C=O); NR^{1b}; C(=O)CR₂R₃; or CR₂R₃CR₄R₅;

R^{1b} and R^{1b′} are independently at each occurrence hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heteroaryl; heteroaralkyl; or heterocyclyl;

R₂, R₃, R₄, R₅ and R₆ are independently at each occurrence hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl or aralkyl;

R^{1c} and R^{1c′} are independently at each occurrence hydrogen; (C₁₋₆)alkyl; aralkyl; aryl; heteroarylalkyl; heteroaryl; heterocyclyl; or together with the nitrogen that they are attached form an aziridine, azetidine, pyrrolidine, piperidine or hexamethyleneimine ring (wherein said aziridine, azetidine, pyrrolidine, piperidine or hexamethyleneimine ring are optionally substiuted with from 1 to 3 substituents selected from halogen, hydroxy; cyano; nitro; (C₁₋₆)alkyl; and aryl);

n, n′ and ″ are independently and at each occurrence integers from 0 to 2;

W₁ and W₂ are each CR₆R₇;

R₇ is independently at each occurrence hydrogen; (C₁₋₆)alkyl; aryl; or heteroaryl;

W₃ and W₄ are each CR₈;

R₈ is independently at each occurrence hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; aralkyl; or R₉;
R₉ is UR^{1d};

U is (CH₂)ₙNR^{1b}(CH₂)_{n′}; (CH₂)ₙNR^{1b}S(O)_{n′}(CH₂)_{n″}; (CH₂)ₙNR^{1b}(C=O)(CH₂)_{n′}; (CH₂)ₙNR^{1b}C(=O)NR^{1b′}(CH₂)_{n′}; (CH₂)ₙNR^{1b}(CO₂)(CH₂)_{n′}; (CH₂)ₙS(CH₂)_{n′}; or (CH₂)ₙO(CH₂)_{n′};
W₅, W₆ and W₇ are independently CR₁₀R₁₁ or NR₁₂;

R₁₀ is independently at each occurrence hydrogen; thiol; (C₁₋₆)alkylthio; halogen; trifluoromethyl; acyloxy; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; or aralkyl;

R₁₁ is independently at each occurrence hydrogen, (C₁₋₆)alkyl; aryl; heteroaryl; or R₉;

R₁₂ is independently at each occurrence hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heteroaryl; heteroaralkyl; heterocyclyl; or R₁₃;

R₁₃ is U′R^{1d};

U′ is (CH₂)ₙ or (C=O)(CR₂R₃)ₙ;

R^{1d} is a substituted or unsubstituted bicyclic carbocyclic or heterocyclic ring system (A):

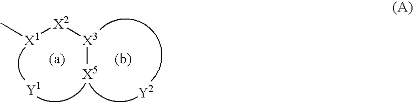

containing up to four heteroatoms in each ring in which at least one of rings (a) and (b) is aromatic;

X¹ is C or N when part of an aromatic ring or CR₁₄ when part of a non aromatic ring;

X² is N, NR₁₅, O, S(O)ₙ, CO or CR₁₄ when part of an aromatic or non-aromatic ring or may in addition be CR₁₆R₁₇ when part of a non aromatic ring;

X³ and X⁵ are independently N or C;

Y¹ is a 0 to 4 atom linker group each atom of which is independently selected from N, NR₁₅, O, S(O)ₙ, CO and CR₁₄ when part of an aromatic or non-aromatic ring or may additionally be CR₁₆R₁₇ when part of a non aromatic ring,

Y² is a 2 to 6 atom linker group, each atom of Y² being independently selected from N, NR₁₅, O, S(O)ₙ, CO and CR₁₄ when part of an aromatic or non-aromatic ring or may additionally be CR₁₆R₁₇ when part of a non aromatic ring;

R₁₄, R₁₆ and R₁₇ are at each occurrence independently selected from: H; (C₁₋₄)alkylthio, halo; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;

R₁₅ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted with (C₁₋₄)alkyl;

or a pharmaceutically acceptable salt or solvate thereof;

provided that the compound of formula (I) contains one R₉ or R₁₃ substituent.

In some embodiments, this invention describes a compound of formula (I) wherein Z₁ and Z₄ are N; and Z₃ is CR^{1a}.

In further embodiments, this invention describes compounds of formula (I) wherein R₁ is OCH₃.

In yet further embodiments, this invention describes compounds of formula (I) wherein R^{1a} is at each occurrence independently hydrogen; halogen; or cyano.

In certain embodiments, this invention describes a compound according to formula (I) wherein AB is CR₂R₃CR₄R₅. In some embodiments where AB is CR₂R₃CR₄R₅; R₂, R₃, R₄ and R₅ are each hydrogen.

In some embodiments of this invention, a compound of formula (I) is described wherein R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅ and W₇ are each CR₁₀R₁₁; R₁₀ is hydrogen; hydroxy; (C₁₋₆)alkyl; acyloxy; or halogen; R₁₁ is hydrogen; (C₁₋₆)alkyl; aryl; or heteroaryl; W₆ is NR₁₂; and R₁₂ is R₁₃.

In yet other embodiments of this invention; a compound of formula (I) is described wherein R₆ is independently at each occurrence hydrogen; hydroxy; halogen; or (C₁₋₆)alkyl; R₇ is independently at each occurrence hydrogen or (C₁₋₆)alkyl; R₈ of W₃ is hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; or aralkyl; R₈ of W₄ is R₉; W₅, W₆ and W₇ are each CR₁₀R₁₁; and R₁₁ is hydrogen; (C₁₋₆)alkyl; aryl; or heteroaryl.

In certain embodiments, this invention describes a compound of formula (I) wherein R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆ and W₇ are each CR₁₀R₁₁; R₁₀ is hydrogen; hydroxy; (C₁₋₆)alkyl; acyloxy; or halogen; R₁₁ of W₅ and W₇ is independently at each occurrence hydrogen; (C₁₋₆)alkyl; aryl; or heteroaryl; and R₁₁ of W6 is R₉.

In some embodiments, this invention describes a compound of formula (I) wherein R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆ and W₇ are each CR₁₀R₁₁; R₁₀ is hydrogen; hydroxy; (C₁₋₆)alkyl; acyloxy; or halogen; R₁₁ of W₅ and W₆ is independently at each occurrence hydrogen; (C₁₆)alkyl; aryl; or heteroaryl; and R₁₁ of W₇ is R₉.

In yet some additional embodiments, this invention describes compounds of formula (I) wherein R^{1d} is 4H-Pyrido[3,2-b][1,4]thiazin-3-oxo-6-yl; 4H-Pyrido[3,2-b]oxazin-3-oxo-6-yl; 2,3-Dihydro-benzo[1,4]dioxin-6-yl; 4H-benzo[1,4]thiazin-3-oxo-6-yl; 2,3-Dihydro-furo[2,3-c]pyridin-5-yl; 4H-Pyrido[3,2-b]oxazin-3-oxo-6-yl; 7-Chloro-4H-pyrido[3,2-b]oxazin-3-oxo-6-yl; 2,3-Dihydro-[1,4]dioxino[2,3-c]-pyridin-6-yl; 2,3-Dihydro-benzofuran-7-carbonitrile-5-yl; 7-Methyl-4H-pyrido[3,2-b][1,4]thiazin-3-oxo-6-yl; 3-Oxa-1-thia-5-aza-indan-5-yl; 5-Methyl-2,3-dihydro-benzo[1,4]dioxin-6-yl; 6-Fluoro-2,3-dihydro[1,4]dioxin-7-yl; 2,3-Dihydro-benzofuran-5-yl; 7-Fluoro-4H-benzo[1,4]thiazin-3-oxo-6-yl; 4H-Benzo[1,4]thiazin-3-oxo-6-yl; or 8-Methyl-2,3-dihydro-benzo[1,4]dioxin-6-yl.

In some embodiments, this invention describes compounds of formula (I) wherein Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆ and W₇ are each CR₁₀R₁₁; R₁₀ is independently at each occurrence hydrogen; hydroxy; (C₁₋₆)alkyl; acyloxy; or halogen; R₁₁ of W₅ and W₆ is independently at each occurrence hydrogen, (C₁₋₆)alkyl; aryl; or heteroaryl; and R₁₁ of W₇ is R₉. In some embodiments of this invention where Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆ and W₇ are each CR₁₀R₁₁; R₁₀ is independently at each occurrence hydrogen; hydroxy; (C₁₋₆)alkyl; acyloxy; or halogen; R₁₁ of W₅ and W₆ is independently at each occurrence hydrogen; (C₁₋₆)alkyl; aryl; or heteroaryl; and R₁₁ of W₇ is R₉; U is (CH₂)ₙNR^{1b}(CH₂)_{n′}; R^{1b} is hydrogen or (C₁₋₆)alkyl; and R^{1d} is 4H-Pyrido[3,2-b][1,4]thiazin-3-oxo-6-yl; 4H-Pyrido[3,2-b]oxazin-3-oxo-6-yl; 2,3-Dihydro-benzo[1,4]dioxin-6-yl; 4H-benzo[1,4]thiazin-3-oxo-6-yl; 2,3-Dihydro-furo[2,3-c]pyridin-5-yl; 4H-Pyrido[3,2-b]oxazin-3-oxo-6-yl; 7-Chloro-4H-pyrido[3,2-b]oxazin-3-oxo-6-yl; 2,3-Dihydro-[1,4]dioxino[2,3-c]-pyridin-6-yl; 2,3-Dihydro-benzofuran-7-carbonitrile-5-yl; 7-Methyl-4H-pyrido[3,2-b][1,4]thiazin-3-oxo-6-yl; 3-Oxa-1-thia-5-aza-indan-5-yl; 5-Methyl-2,3-dihydro-benzo[1,4]dioxin-6-yl; 6-Fluoro-2,3-dihydro[1,4]dioxin-7-yl; 2,3-Dihydro-benzofuran-5-yl; 7-Fluoro-4H-benzo[1,4]thiazin-3-oxo-6-yl; 4H-Benzo[1,4]thiazin-3-oxo-6-yl; or 8-Methyl-2,3-dihydro-benzo[1,4]dioxin-6-yl.

In some embodiments, this invention describes a compound of formula (I) wherein Z₁ and Z₄ are N; Z₃ is CR^{1a}; R¹ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆ and W₇ are each CR₁₀R₁₁; R₁₀ is independently at each occurrence hydrogen; hydroxy; (C₁₋₆)alkyl; acyloxy; or halogen; R₁₁ of W₅ and W₇ is independently at each occurrence hydrogen, (C₁₋₆)alkyl; aryl; or heteroaryl; and R₁₁ of W₆ is R₉. In some embodiments Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅, W₆and W₇ are each CR₁₀R₁₁; R₁₀ is independently at each occurrence hydrogen; hydroxy; (C₁₋₆)alkyl; acyloxy; or halogen; R₁₁ of W₅ and W₇ is independently at each occurrence hydrogen; (C₁₋₆)alkyl; aryl; or heteroaryl; and R₁₁ of W6 is R₉; U is (CH₂)ₙNR^{1b}(CH₂)_{n′}; R^{1b} is hydrogen or (C₁₋₆)alkyl; and R^{1d} is 4H-Pyrido[3,2-b][1,4]thiazin-3-oxo-6-yl; 4H-Pyrido[3,2-b]oxazin-3-oxo-6-yl; 2,3-Dihydro-benzo[1,4]dioxin-6-yl; 4H-benzo[1,4]thiazin-3-oxo-6-yl; 2,3-Dihydro-furo[2,3-c]pyridin-5-yl; 4H-Pyrido[3,2-b]oxazin-3-oxo-6-yl; 7-Chloro-4H-pyrido[3,2-b]oxazin-3-oxo-6-yl; 2,3-Dihydro-[1,4]dioxino[2,3-c]-pyridin-6-yl; 2,3-Dihydro-benzofuran-7-carbonitrile-5-yl; 7-Methyl-4H-pyrido[3,2-b][1,4]thiazin-3-oxo-6-yl; 3-Oxa-1-thia-5-aza-indan-5-yl; 5-Methyl-2,3-dihydro-benzo[1,4]dioxin-6-yl; 6-Fluoro-2,3-dihydro[1,4]dioxin-7-yl; 2,3-Dihydro-benzofuran-5-yl; 7-Fluoro-4H-benzo[1,4]thiazin-3-oxo-6-yl; 4H-Benzo[1,4]thiazin-3-oxo-6-yl; or 8-Methyl-2,3-dihydro-benzo[1,4]dioxin-6-yl.

In some embodiments, this invention describes a compound of formula (I) wherein Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ of W₃ is hydrogen; (C₁₋₆)alkyl; hydroxy; or halogen; R₈ of W₄ is R₉; W₅, W₆ and W₇ are each CR₁₀R₁₁; R₁₀ is independently at each occurrence hydrogen; hydroxy; (C₁₋₆)alkyl; acyloxy; or halogen; and R₁₁ is independently at each occurrence hydrogen; (C₁₋₆)alkyl; aryl; or heteroaryl. In some further embodiments, this invention describes a compound of formula (I) wherein Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ of W₃ is hydrogen; (C₁₋₆)alkyl; hydroxy; or halogen; R₈ of W₄ is R₉; W₅, W₆ and W₇ are each CR₁₀R₁₁; R₁₀ is independently at each occurrence hydrogen; hydroxy; (C₁₋₆)alkyl; acyloxy; or halogen; and R₁₁ is independently at each occurrence hydrogen; (C₁₋₆)alkyl; aryl; or heteroaryl, and U is (CH₂)ₙNR^{1b}(CH₂)_{n′}; R^{1b} is hydrogen or (C₁₋₆)alkyl; R^{1d} is 4H-Pyrido[3,2-b][1,4]thiazin-3-oxo-6-yl; 4H-Pyrido[3,2-b]oxazin-3-oxo-6-yl; 2,3-Dihydro-benzo[1,4]dioxin-6-yl; 4H-benzo[1,4]thiazin-3-oxo-6-yl; 2,3-Dihydro-furo[2,3-c]pyridin-5-yl; 4H-Pyrido[3,2-b]oxazin-3-oxo-6-yl; 7-Chloro-4H-pyrido[3,2-b]oxazin-3-oxo-6-yl; 2,3-Dihydro-[1,4]dioxino[2,3-c]-pyridin-6-yl; 2,3-Dihydro-benzofuran-7-carbonitrile-5-yl; 7-Methyl-4H-pyrido[3,2-b][1,4]thiazin-3-oxo-6-yl; 3-Oxa-1-thia-5-aza-indan-5-yl; 5-Methyl-2,3-dihydro-benzo[1,4]dioxin-6-yl; 6-Fluoro-2,3-dihydro[1,4]dioxin-7-yl; 2,3-Dihydro-benzofuran-5-yl; 7-Fluoro-4H-benzo[1,4]thiazin-3-oxo-6-yl; 4H-Benzo[1,4]thiazin-3-oxo-6-yl; or 8-Methyl-2,3-dihydro-benzo[1,4]dioxin-6-yl.

In some embodiments, this invention describes compounds of formula (I) wherein; Z₁ and Z₄ are N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₆)alkyl; hydroxy; or halogen; W₅ and W₇ are each CR₁₀R₁₁; R₁₀ is independently selected from hydrogen; hydroxy; (C₁₋₆)alkyl; acyloxy; or halogen; R₁₁ is independently at each occurrence hydrogen; (C₁₋₆)alkyl; aryl; or heteroaryl; W₆ is NR₁₂; and R₁₂ is R₁₃. In some further embodiments, this invention describes compounds of formula (I) wherein; Z₁ and Z₄ are. N; Z₃ is CR^{1a}; R₁ is OCH₃; R^{1a} is at each occurrence independently hydrogen; halogen; or cyano; AB is CH₂CH₂; R₆ is independently at each occurrence hydrogen; hydroxy; halogen; or (C₁₋₆)alkyl; R₇ is independently at each occurrence hydrogen or (C₁₋₆)alkyl; R₈ is independently at each occurrence hydrogen; (C₁₋₆)alkyl; hydroxy; or halogen; W₅ and W₇ are each CR₁₀R₁₁; R₁₀ is independently selected from hydrogen; hydroxy; (C₁₋₆)alkyl; acyloxy; or halogen; R₁₁ is independently at each occurrence hydrogen; (C₁₋₆)alkyl; aryl; or heteroaryl; W₆ is NR₁₂; and R₁₂ is R₁₃; and U′ is (CH₂)ₙ; and R^{1d} is 4H-Pyrido[3,2-b][1,4]thiazin-3-oxo-6-yl; 4H-Pyrido[3,2-b]oxazin-3-oxo-6-yl; 2,3-Dihydro-benzo[1,4]dioxin-6-yl; 4H-benzo[1,4]thiazin-3-oxo-6-yl; 2,3-Dihydro-furo[2,3-c]pyridin-5-yl; 4H-Pyrido[3,2-b]oxazin-3-oxo-6-yl; 7-Chloro-4H-pyrido[3,2-b]oxazin-3-oxo-6-yl;2,3-Dihydro-[1,4]dioxino[2,3-c]-pyridin-6-yl; 2,3-Dihydro-benzofuran-7-carbonitrile-5-yl; 7-Methyl-4H-pyrido[3,2-b][1,4]thiazin-3-oxo-6-yl; 3-Oxa-1-thia-5-aza-indan-5-yl; 5-Methyl-2,3-dihydro-benzo[1,4]dioxin-6-yl; 6-Fluoro-2,3-dihydro[1,4]dioxin-7-yl; 2,3-Dihydro-benzofuran-5-yl; 7-Fluoro-4H-benzo[1,4]thiazin-3-oxo-6-yl;4H-Benzo[1,4]thiazin-3-oxo-6-yl; or 8-Methyl-2,3-dihydro-benzo[1,4]dioxin-6-yl.

In certain aspects, this invention describes a compound of the formula (±)-6-{[((3aR,4R,6aS)-2-{2-[3-fluoro-6-(methyloxy)-1,5-naphthyridin-4-yl]ethyl}octahydrocyclopenta[c]pyrrol-4-yl)amino]methyl}-2H-pyrido[3,2-b][1,4]thiazin-3(4H)-one; (±)-6-{[((3aR,4S,6aS)-2-{2-[3-fluoro-6-(methyloxy)-1,5-naphthyridin-4-yl]ethyl}octahydrocyclopenta[c]pyrrol-4-yl)amino]methyl}-2H-pyrido[3,2-b][1,4]thiazin-3(4h)-one; 6-{[((3aR,6aS)-2-{2-[3-fluoro-6-(methyloxy)-1,5-naphthyridin-4-yl]ethyloctahydrocyclopenta[c]pyrrol-5-yl)amino]methyl}-2H-pyrido3(4H)-one; (±)-6-{[(3aR,6aS)-5-{2-[3-fluoro-6-(methyloxy)-1,5-naphthyridin-4-yl]ethyl}hexahydropyrrolo[3,4-c]pyrrol-2(1H)-yl]methyl}-2H-pyrido[3,2-b][1,4]thiazin-3(4M-one; or (±)-6-[({[(3aS,6aR)-2-{2-[3-fluoro-6-(methyloxy)-1,5-naphthyridin-4-yl]ethyl}hexahydrocyclopenta[c]pyrrol-3a(1H)-yl]methyl}amino)methyl]-2H-pyrido[3,2-b][1,4]thiazin-3(4H)-one; or a pharmaceutically acceptable salt or solvate thereof.

In certain aspects, this invention describes a process for the preparation of compounds of formula (I) (wherein AB is CR₂R₃CR₄R₅), which process comprises:

(a) reacting a compound of formula (a) with a compound of formula (b) to give a compound of formula (I): 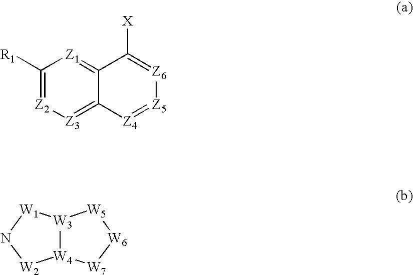
wherein X is CH=CH₂ or AB(CH₂)ₙ-L;

L is a leaving group; and

Z₁, Z₂, Z₃, Z₄, Z₅ Z₆, R₁, W₁, W₂, W₃, W₄, W₅, W₆, W₇ and n are as defined in formula (I).

In some embodiments, this invention describes a process for the preparation of compounds of formula (I) wherein (wherein AB is CR₂R₃CR₄R₅), which process comprises:

(a) reacting a compound of formula (a) with a compound of formula (b′) to form compound (c);

(b) removing P, P′ or P″ from (c) where P, P′ or P″ is not hydrogen;

(c) reacting a compound of formula (c) with a compound of formula (d), (e) or (f) to form a compound of formula (I) of claim 5; 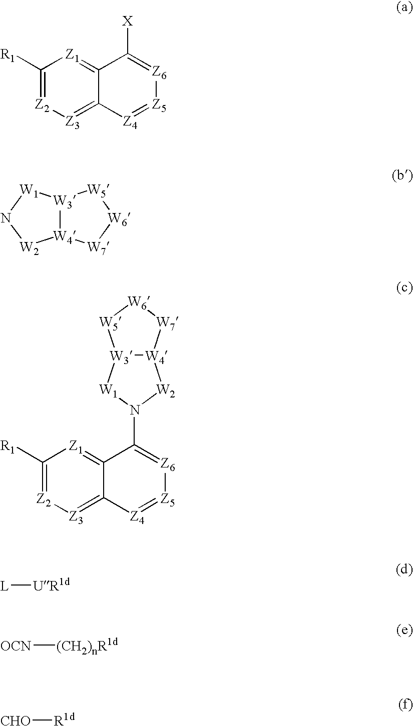
wherein:

X is CH=CH₂ or AB(CH₂)ₙ-L;

Z₁, Z₂, Z_{3,} Z_{4,} Z₅ Z₆, AB, n, n′, R^{1b}, R^{1c}, R^{1c′}, W₁, W₂, R₁, and R₁₀ are as described in claim 5;

L is independently at each occurrence a leaving group;

W₃′ and W₄′ are CR₈′;

R₈′ is independently at each occurrence hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonl; aralkyl; aryl; heteroarylalkyl; heteroaryl; heterocyclyl; hydroxy; amino; NR^{1c}CR^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; aralkyl; or R₉′;

R₉′ is (CH₂)ₙNR^{1b}P, SP′ or OP″;

P is hydrogen or a nitrogen protecting group;

P′ is hydrogen or a sulphur protecting group;

P″ is hydrogen or an oxygen protecting group;

W₅′, W₆′ and W₇′ are independently CR₁₀R₁₁′ or NR₁₂′;

R₁₁′ is hydrogen, (C₁₋₆)alkyl; aryl; heteroaryl; or R₉′;

R₁₂ is hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heteroaryl; heteroaralkyl; heterocyclyl; or R₁₃′;

R₁₃′ is hydrogen or P; and

U″ is (CH₂)ₙ; S(O)ₙ(CH₂)_{n′}; (O=C)(CR₂CR₃)ₙ; or (O=C)O(CH₂)ₙ;

provided that the compound of formula (c) contains exactly one R₉′ or R₁₃′ substituent.

In some aspects, this invention describes a pharmaceutical composition comprising a compound according to formula (I) or any of the embodiments described herein, and a pharmaceutically acceptable carrier.

In certain embodiments, this invention describes a method of treating bacterial infections in mammals which comprises administering to a mammal in need thereof an effective amount of a compound according to formula (I) or any of its embodiments described herein.

Also included in the ambit of this invention are pharmaceutically acceptable addition salts, solvates or prodrugs of the compounds of this invention. Prodrugs are considered to be any covalently bonded carriers which release the active parent drug according to formula (I) in vivo.

Unless otherwise defined, the term “alkyl” when used alone or when forming part of other groups (such as the ‘alkoxy’ group) includes substituted or unsubstituted, straight or branched chain alkyl groups containing the specified range of carbon atoms. For example, the term “(C₁₋₆)alkyl” include methyl, ethyl, propyl, butyl, iso-propyl, sec-butyl, tert-butyl, iso-pentyl, and the like.

The term “alkenyl” means a substituted or unsubstituted alkyl group of the specified range of carbon atoms, wherein one carbon-carbon single bond is replaced by a carbon-carbon double bond. For example, the term “(C₂₋₆)alkenyl” include ethylene, 1-propene, 2-propene, 1-butene, 2-butene, and isobutene, and the like. Both cis and trans isomers are included.

The term “cycloalkyl” refers to substituted or unsubstituted carbocyclic system of the specified range of carbon atoms, which may contain up to two unsaturated carbon-carbon bonds. For example, the term “(C₃₋₇)cycloalkyl” include cyclopropyl, cyclobutyl, cyclopentyl, cyclopentenyl, cyclohexyl, cyclohexenyl, and cycloheptyl.

The term “alkoxy” refers to an O-alkyl radical where the alkyl group contains the specified range of carbon atoms and is as defined herein.

The term “acyl” refers to a C(=O)alkyl or a C(=O)aryl radical. In some embodiments, the alkyl group contains 13 or less carbons; in some embodiments 10 or less carbon atoms; in some embodiments 6 or less carbon atoms; and is as otherwise defined. Aryl is as defined herein.

The term “alkylsulphonyl” refers to a SO₂alkyl radical wherein the alkyl group contains the specified range of carbon atoms and is as defined herein.

The term “alkylthio” refers to a Salkyl the alkyl group contains the specified range of carbon atoms and is as defined herein.

The term “aralkyl” refers to an alkyl radical, wherein said alkyl radical contains 13 carbons or less and more preferably 6 carbons or less, which is joined to an aryl group, where aryl is as otherwise defined. The alkyl group may be branched or straight chain, and the aryl group maybe joined to any primary, secondary or tertiary carbon of said alkyl chain.

The term “heteroaralkyl” refers to an alkyl radical, wherein said alkyl radical contains 13 carbons or less and more preferably 6 carbons or less, which is joined to a heteroaryl group, where heteroaryl is as otherwise defined. The alkyl group may be branched or straight chain, and the heteroaryl group maybe joined to a primary, secondary or tertiary carbon of said alkyl chain.

The term “heterocyclylthio” refers to an S-heterocyclyl radical wherein the heterocyclyl moiety is as defined herein.

The term “heterocyclyloxy” refers to an O-heterocyclyl radical wherein heterocyclyl is as defined herein.

The term “arylthio” refers to an S-aryl radical wherein aryl is as defined herein.

The term “aryloxy” refers to an O-aryl radical wherein aryl is as defined herein.

The term “acylthio” refers to an S-acyl radical wherein acyl is as defined herein.

The term “acyloxy” refers to an O-acyl radical wherein acyl is as defined herein.

The term “alkoxycarbonyl” refers to a CO₂alkyl radical wherein the alkyl group contains the specified range of carbon atoms and is as defined herein.

The term “alkylsulphonyloxy” refers to an O-SO₂alkyl radical wherein the alkyl group contains the specified range of carbon atoms and is as defined herein.

The term “arylsulphonyl” refers to a SO₂aryl radical wherein aryl is as herein defined.

The term “arylsulphoxide” refers to a SOaryl radical wherein aryl is as defined herein.

Unless otherwise defined, suitable substituents for any alkyl, alkoxy, alkenyl, and cycloalkyl groups includes up to three substituents selected from the group consisting of hydroxy, halogen, nitro, cyano, carboxy, amino, amidino, sulphonamido, unsubstituted (C₁₋₃)alkoxy, trifluromethyl, and acyloxy.

Halo or halogen includes fluoro, chloro, bromo and iodo.

The term “haloalkyl” refers to an alkyl radical containing the specified range of carbon atoms and is as otherwise defined herein, which is further substituted with 1-3 halogen atoms.

The term “haloalkoxy” refers to an alkoxy radical of the specified range and as defined herein, which is further substituted with 1-3 halogen atoms.

The term “hydroxyalkyl” refers to an alkyl group as defined herein, further substituted with a hydroxy group.

Unless otherwise defined, the term “heterocyclic” as used herein includes optionally substituted aromatic and non-aromatic, single and fused, mono- or bicyclic rings suitably containing up to four hetero-atoms in each ring selected from oxygen, nitrogen and sulphur, which rings may be unsubstituted or C-substituted by, for example, up to three groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; hydroxy, (C₁₋₄)alkyl; (C₁₋₄)thioalkyl; (C₁₋₄)alkoxy; nitro; cyano, carboxy; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl.

Each heterocyclic ring suitably has from 3 to 7, preferably 5 or 6, ring atoms. A fused heterocyclic ring system may include carbocyclic rings and need include only one heterocyclic ring.

Compounds within the invention containing a heterocyclyl group may occur in two or more tautometric forms depending on the nature of the heterocyclyl group; all such tautomeric forms are included within the scope of the invention.

Where an amino group forms part of a single or fused non-aromatic heterocyclic ring as defined above suitable optional substituents in such substituted amino groups include hydrogen; trifluoromethyl; (C₁₋₄)alkyl optionally substituted by hydroxy, (C₁₋₄)alkoxy, (C₁₋₄)alkylthio, halo or trifluoromethyl; and (C₂₋₄)alkenyl.

When used herein the term “aryl”, includes optionally substituted phenyl and naphthyl.

Aryl groups may be optionally substituted with up to five, preferably up to three, groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; (C₁₋₄)hydroxyalkyl; (C₁₋₄)alkylthio; (C₁₋₄)alkoxy; nitro; cyano; carboxy; amino or aminocarbonyl optionally substituted by (C₁₋₄)alkyl; (C₁₋₄)alkylsulphonyl; (C₂₋₄)alkenylsulphonyl.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Furthermore, it will be understood that phrases such as “a compound of Formula I or a pharmaceutically acceptable salt, solvate or derivative thereof” are intended to encompass the compound of Formula I, a derivative of formula (I), a pharmaceutically acceptable salt of the compound of formula (I), a solvate of formula (I), or any pharmaceutically acceptable combination of these. Thus by way of non-limiting example used here for illustrative purpose, “a compound of Formula I or a pharmaceutically acceptable salt or solvate thereof” may include a pharmaceutically acceptable salt of a compound of formula (I) that is further present as a solvate.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable salts of the above-mentioned compounds of formula (I) include the free base form or their acid addition or quaternary ammonium salts, for example their salts with mineral acids e.g. hydrochloric, hydrobromic, sulphuric nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. Compounds of formula (I) having a free carboxy group may also be prepared as an in vivo hydrolysable ester. The invention extends to all such derivatives. One of skill in the art will recognize that where compounds of the invention contain multiple basic sites, a compound of the invention maybe present as a salt complexed with more than one equivalent of a corresponding acid or mixture of acids.

Pharmaceutically acceptable derivatives refers to compounds of formula (I) that have been covalently modifed with a group that undergoes at least some in vivo cleavage to a compound of formula (I).

Examples of suitable pharmaceutically acceptable in vivo hydrolysable ester-forming groups include those forming esters which break down readily in the human body to leave the parent acid or its salt.

Suitable groups of this type include those of part formulae (i), (ii), (iii), (iv) and (v):

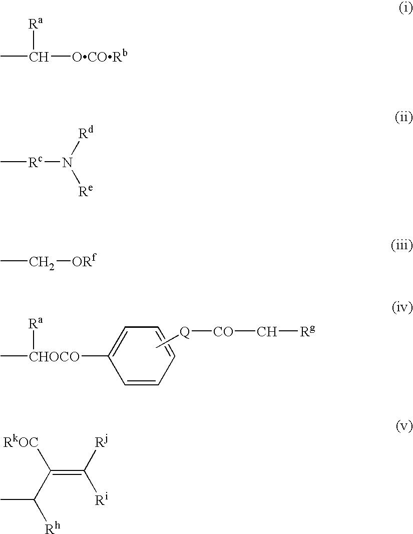

wherein R^{a} is hydrogen, (C₁₋₆) alkyl, (C₃₋₇) cycloalkyl, methyl, or phenyl, R^{b} is (C₁₋₆) alkyl, (C₁₋₆)alkoxy, phenyl, benzyl, (C₃₋₇)cycloalkyl, (C₃₋₇)cycloalkyloxy, (C₁₋₆)alkyl(C₃₋₇) cycloalkyl, 1-amino(C₁₋₆)alkyl, or

1-(C₁₋₆ alkyl)amino(C₁₋₆) alkyl; or R^{a} and R^{b} together form a 1,2-phenylene group optionally substituted by one or two methoxy groups; R^{c} represents (C₁₋₆)alkylene optionally substituted with a methyl or ethyl group and R^{d} and R^{e} independently represent (C₁₋₆) alkyl; R^{f} represents (C₁₋₆) alkyl; R^{g} represents hydrogen or phenyl optionally substituted by up to three groups selected from halogen, (C₁₋₆) alkyl, or (C₁₋₆) alkoxy; Q is oxygen or NH; R⁹ is hydrogen or

(C₁₋₆) alkyl; Rⁱ is hydrogen, (C₁₋₆) alkyl optionally substituted by halogen, (C₂₋₆) alkenyl, (C₁₋₆)alkoxycarbonyl, aryl or heteroaryl; or R^{h} and Rⁱ together form (C₁₋₆) alkylene; R^{j} represents hydrogen, (C₁₋₆) alkyl or (C₁₋₆)alkoxycarbonyl; and R^{k} represents (C₁₋₈)alkyl, (C₁₋₈)alkoxy, (C₁₋₆)alkoxy(C₁₋₆)alkoxy or aryl.

Examples of suitable in vivo hydrolysable ester groups include, for example, acyloxy(C₁₋₆)alkyl groups such as acetoxymethyl, pivaloyloxymethyl, acetoxyethyl, pivaloyloxyethyl, 1-(cyclohexylcarbonyloxy)prop-1-yl, and (1-aminoethyl)carbonyloxymethyl; (C₁₋₆)alkoxycarbonyloxy(C₁₋₆)alkyl groups, such as ethoxycarbonyloxymethyl, ethoxycarbonyloxyethyl and propoxycarbonyloxyethyl; di(C₁₋₆)alkylamino(C₁₋₆)alkyl especially di(C₁₋₄)alkylamino(C₁₋₄)alkyl groups such as dimethylaminomethyl, dimethylaminoethyl, diethylaminomethyl or diethylaminoethyl; 2-(C₁₋₆)alkoxycarbon-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolysable ester-forming group is that of the formula:

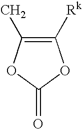

wherein R^{k} is hydrogen, C₁₋₆alkyl or phenyl.

R is preferably hydrogen.

Compounds of formula (I) may also be prepared as the corresponding N-oxides.

Certain of the compounds of formula (I) may exist in the form of optical isomers, e.g. diastereoisomers and mixtures of isomers in all ratios, e.g. racemic mixtures. The invention includes all such form, including pure isomeric forms. The different isomeric forms may be separated or resolved one from the other by conventional methods, or any given isomer may be obtained by conventional synthetic methods or by stereospecific or asymmetric syntheses.

One of skill in the art should interpret the term “reaction” to indicate the means used for effecting a desired transformation as indicated by the context such term is used. One of skill in the art further appreciates that what constitutes “reacting” for purposes of this invention, requires the use of conditions necessary to bring about the desired outcome. Accordingly, one of skill in the readily appreciates that optimization for a given reaction may require some variation in reaction parameters such as reaction time, temperature, energy source, pressure, light, pressure, solvent or solvents used, reactants, reagents, co-reagents, catalysts, and the like.

Protective groups wherever found herein maybe designated by their specific formula or alternatively, maybe referred to generically by P or Pₙ (wherein n is an integer). It is to be appreciated that where generic descriptors are used, that such descriptors are at each occurrence independent from each other. Thus, a compound with more than one of the same generic descriptors (e.g. P) does not indicate that each P is the same protective group, they maybe the same or different, so long as the group is suitable to the chemistry being employed. Where protection or deprotection is generically referred to, one of ordinary skill in the art will understand this to mean that suitable conditions are employed that will allow for the removal of the protecting group to be removed while minimizing reaction at other positions of the molecule, unless otherwise indicated. Many protective groups and protective group strategies are known to those of skill in the art in maybe found in numerous references including, Greene, et al. “Protective Groups in Organic Synthesis” (Published by Wiley-Interscience), which is herein incorporated by reference in its entirety.

Leaving groups wherever found herein maybe designated by a specific chemical formula, or alternatively, maybe generically referred to as L or Ln (wherein n is an integer). It is to be appreciated that where a generic descriptor is used, that such descriptors are at each occurrence independent from each other. Leaving groups can be single atoms such as Cl, Br, or I, or maybe a group such as OSO₂CH₃, OC(=O)CH₃, O(C=O)CF₃, OSO₂CF₃, and the like. One skilled in the art will readily ascertained that leaving groups generally refer to atoms or groups which can be eliminated, substituted or otherwise dissociate during the course of the reaction.

The antibacterial compounds according to the invention may be formulated for administration in any convenient way for use in human or veterinary medicine, by analogy with other antibacterials.

The pharmaceutical compositions of the invention include those in a form adapted for oral, topical or parenteral use and may be used for the treatment of bacterial infection in mammals including humans.

The composition may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tablefting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl phydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

No toxicological effects are indicated when a compound of formula (I) or a pharmaceutically acceptable derivative thereof is administered in the above-mentioned dosage range.

The compound of formula (I) may be the sole therapeutic agent in the 5 compositions of the invention or a combination with other antibacterials. If the other antibacterial is a β-lactam then a β-lactamase inhibitor may also be employed.

Compounds of formula (I) are active against a wide range of organisms including both Gram-negative and Gram-positive organisms.

All publications, including but not limited to patents and patent applications, cited in this specification are herein incorporated by reference (whether specifically stated to be so or not) as if each individual publication were specifically and individually indicated to be incorporated by reference herein as though fully set forth.

The following examples illustrate the preparation of certain compounds of formula (I) and the activity of certain compounds of formula (I) against various bacterial organisms.

The examples of the present invention were prepared by the methods illustrated in Schemes I through IV. 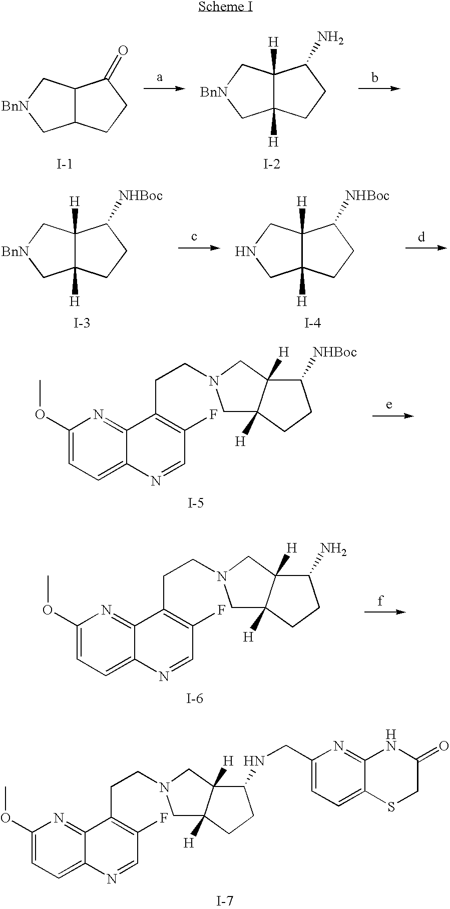
Reagents and conditions:

(a) hydroxylamine, DCM-MeOH (1:10), 10% NaOH in H₂O, 50° C.; then LAH, THF, reflux (b) Boc₂O, THF, 25° C. (c) H₂ (50 psi), 30% Pd(OH)₂ (wet), EtOH (d) 8-ethenyl-7-fluoro-2-(methyloxy)-1,5-naphthyridine, EtOH, 85° C. (e) 4N HCl in dioxane, MeOH, 25° C. (f) 3-oxo-3,4-dihydro-2H-pyrido[3,2-b][1,4]thiazine-6-carbaldehyde, DCM-EtOH (1:1), Na₂SO₄, then NaBH₄, 25° C.

2-(phenylmethyl)hexahydrocyclopenta[c]pyrrol-4(1H)-one (I-1) was prepared according to the procedure of Achiwa, L.; et al. Chem. Pharm. Bull. 1985, 33, 2762. The ketone was transformed into amine (1-2) via hydroxylamine formation and subsequent reduction generating a mixture of diastereomers. The amine of each diastereomer was protected as the Boc carbamate. Hydrogenation removed the benzyl group and successive Michael addition of the amine into 8-ethenyl-7-fluoro-2-(methyloxy)-1,5-naphthyridine generated the adduct (I-5). The Boc group was removed and the resulting free amine (I-6) was reacted with 3-oxo-3,4-dihydro-2H-pyrido[3,2-b][1,4]thiazine-6-carbaldehyde, providing the final compound (I-7). 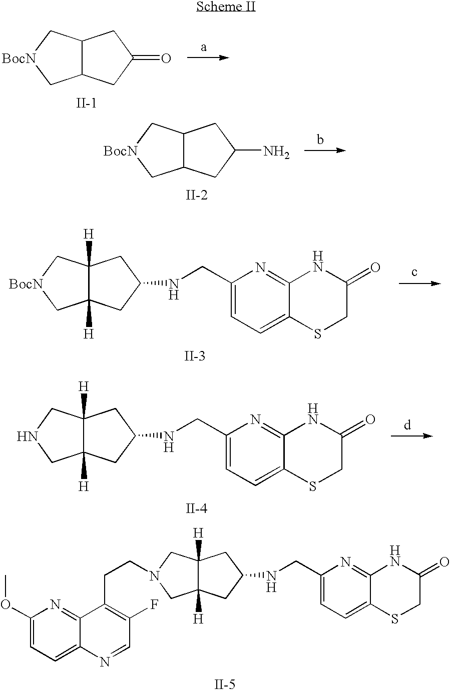
Reagents and conditions:

(a) HCO₂NH₄, NaCNBH₃, MeOH, 25° C. (b) 3-oxo-3,4-dihydro-2H-pyrido[3,2-b][1,4]thiazine-6-carbaldehyde, DCM-EtOH (1:1), Na₂SO₄, then NaBH₄, 25° C. (c) 4N HCl in dioxane, MeOH, 25° C. (d) 8-ethenyl-7-fluoro-2-(methyloxy)-1,5-naphthyridine, EtOH, 85° C.

1,1-dimethylethyl 5-oxohexahydrocyclopenta[c]pyrrole-2(1H)-carboxylate (II-1) was prepared according to Becker, D. P.; Flynn, D. L.; Tetrahedron, 1993, 49, 23, 5049. The ketone underwent reductive amination with ammonium formate forming the free amine (II-2), which was then reacted through a second reductive amination with 3-oxo-3,4-dihydro-2H-pyrido[3,2-b][1,4]thiazine-6-carbaldehyde yielding II-3. The Boc group was removed and subsequent Michael addition into the vinyl naphthyridine, as described in Scheme I, provided the final compound (II-5). 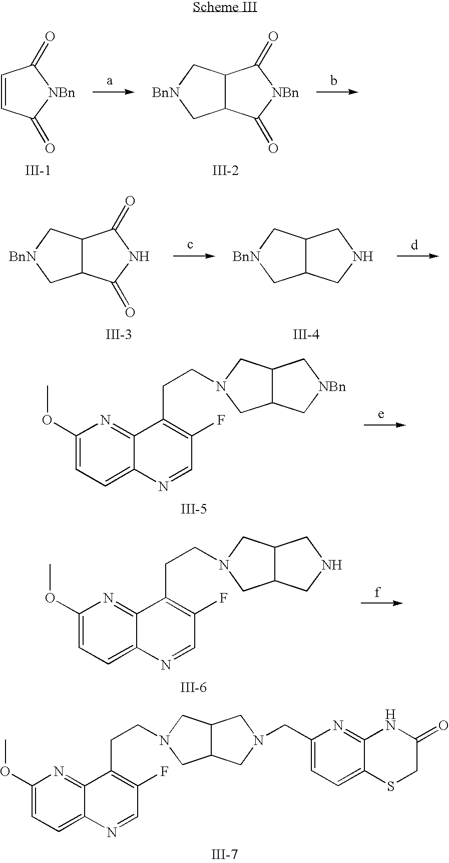
Reagents and conditions:

(a) [(methyloxy)methyl](phenylmethyl)[(trimethylsilyl)methyl]amine, DCM, TFA (b) H₂ (50 psi), 10% Pd-C, EtOH (c) LAH, THF, 0-25° C. (d) 8-ethenyl-7-fluoro-2-(methyloxy)-1,5-naphthyridine, EtOH, 85° C. (e) H₂ (50 psi), 30% Pd(OH)₂ (wet), EtOH (f) 3-oxo-3,4-dihydro-2H-pyrido[3,2-b][1,4]thiazine-6-carbaldehyde, DCM, Na₂SO₄, then NaBH(OAc)₃, 25° C.

Azomethine ylide cyloaddition onto 1-(phenylmethyl)-1H-pyrrole-2,5-dione yielded the succinimide (III-2). The benzyl group of the succinimide was selectively removed through hydrogenolysis and subsequent carbonyl reduction provided the free amine (III-4). Michael addition of the amine into 8-ethenyl-7-fluoro-2-(methyloxy)-1,5-naphthyridine afforded adduct (III-5). The remaining secondary amine was liberated through exhaustive hydrogenolysis, where subsequent reductive amination with 3-oxo-3,4-dihydro-2H-pyrido[3,2-b][1,4]thiazine-6-carbaldehyde provided the final compound (III-7). 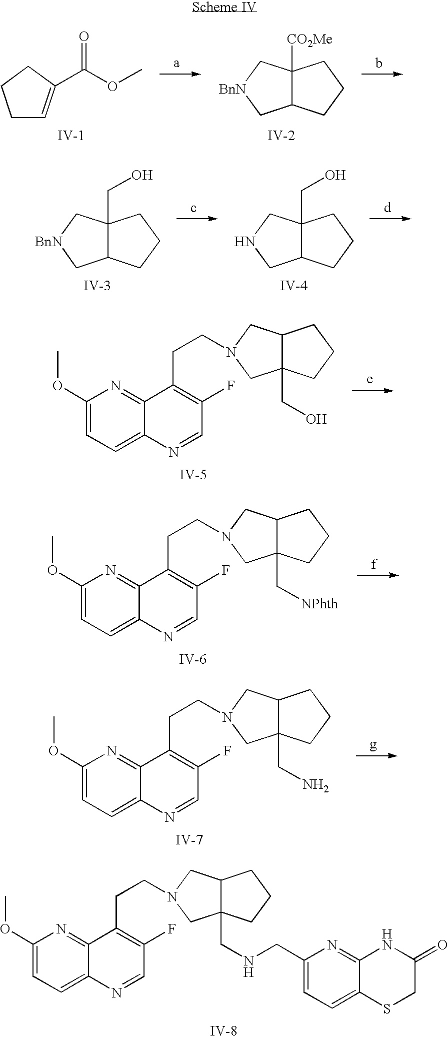
Reagents and conditions:

(a) [(methyloxy)methyl](phenylmethyl)[(trimethylsilyl)methyl]amine, DCM, TFA (b) LAH, THF, 0° C. (c) H₂ (50 psi), 30% Pd(OH)₂ (wet), EtOH (d) 8-ethenyl-7-fluoro-2-(methyloxy)-1,5-naphthyridine, EtOH, 85° C. (e) phthalimide, DEAD, PPh₃, THF, 70° C. (f) NH₂NH₂, EtOH, reflux (g) 3-oxo-3,4-dihydro-2H-pyrido[3,2-b][1,4]thiazine-6-carbaldehyde, DCM-EtOH (1:1), Na₂SO₄, then NaBH₄, 25° C.

Azomethine ylide cycloaddition onto methyl 1-cyclopentene-1-carboxylate (IV-1), afforded methyl ²-(phenylmethyl)hexahydrocyclopenta[c]pyrrole-3a(1H)-carboxylate (IV-2). The ester was reduced to the alcohol and the benzyl group removed through hydrogenolysis providing the free amine (IV-4). Subsequent Michael addition into 8-ethenyl-7-fluoro-2-(methyloxy)-1,5-naphthyridine yielded adduct (IV-5). The hydroxyl group was replaced with phthalimide using the standard Mitsunobu conditions and the primary amine (IV-7) was unmasked using hydrazine. Reaction of the amine with 3-oxo-3,4-dihydro-2H-pyrido[3,2-b][1,4]thiazine-6-carbaldehyde under the standard reductive amination conditions provided the final compound (IV-8).

### General

Proton nuclear magnetic resonance (¹H NMR) spectra were recorded at 400 MHz, and chemical shifts are reported in parts per million (δ) downfield from the internal solvent standard CHCl₃ or MeOH. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDl₃ is deuteriochloroform and CD₃OD is tetradeuteriomethanol. Mass spectra were obtained using electrospray (ES) ionization techniques. All temperatures are reported in degrees Celsius. E. Merck Silica Gel 60 F-254 thin layer plates were used for thin layer chromatography. Flash chromatography was carried out on E. Merck Kieselgel 60 (230-400 mesh) silica gel. Analytical HPLC was performed on Beckman chromatography systems. Preparative HPLC was performed using Gilson chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

All compounds listed below were formed as racemic mixtures, illustrated stereochemistry is relative only (defines diastereomeric relationships).

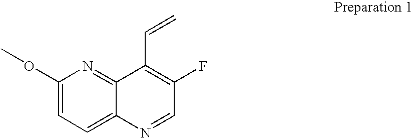

### Preparation of 8-ethenyl-7-fluoro-2-(methyloxy)-1,5-naphthyridine

(a) (2-[(6-Methoxypyridin-3-ylamino)methylene]malonic acid diethyl ester

A solution of 5-amino-2-methoxypyridine (Aldrich, 100 g, 0.806 mole) and diethyl ethoxymethylenemalonate (Aldrich, 163 mL, 0.806 mole) in EtOH (1 L) was heated at reflux for 4 h, then was cooled to RT. Concentration to dryness gave the title compound (238 g, quantitative).

(b) 6-Methoxy-4-oxo-1,4-dihydro-[1,5]naphthyridine-3-carboxylic acid ethyl ester

Dowtherm A (Fluka, 500 mL) was brought to boiling (250° C.) in a 2 L 3-neck flask fitted with a still-head and a reflux condenser. 2-[(6-Methoxypyridin-3-ylamino)methylene]malonic acid diethyl ester (100 g, 0.34 mole) was added portionwise over 5 min. The solution was heated at reflux for an additional 15 min, allowing some solvent to distil over. The resulting solution was cooled to 25° C. and diluted with hexanes (750 mL). The mixture was cooled in ice for 1 hr, then the brown solid was filtered off, washed with hexanes, and dried under vacuum to afford the title compound (61.72g, 73%).

(c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (74.57 g, 300 mmole) in dry DMF (260 mL) under argon was stirred vigorously in a water bath (to maintain approximately RT—may need slight ice-cooling on a large scale). Phosphorus tribromide (30.0 mL, 316 mmole) was added dropwise over 15 min and stirring was continued for an additional 30 min. Water (1 L) was added, followed by saturated sodium carbonate solution to pH 7. The solid was collected by suction filtration, washed with water and dried under vacuum over phosphorus pentoxide to give the title compound (83.56 g, 90%).

(d) 4-Bromo-6-methoxy-[1,5]naphthyridine-3-carboxylic acid

2 N NaOH (300 mL, 600 mmol) was added dropwise over 30 min to a stirred solution of 4-bromo-6-methoxy-[1,5]naphthyridine-3-carboxylic acid ethyl ester (83.56 g, 268 mmol) in THF (835 mL). Stirring was continued overnight, at which time LC/IMS showed that the saponification was complete. 2 N HCl was added to pH 6 and the THF was removed in vacuo. 2 N HCl was added to pH 2, then water (250 mL) was added, and the mixture was cooled thoroughly in ice. The solid was collected by suction filtration, washed with water and dried (first using a rotary evaporator at 50° C. and then under high vacuum at 50° C. overnight) to give the title compound (76.7 g, slightly over quantitative). This material was used without further purification.

(e) 4-Bromo-6-methoxy-[1,5]naphthyridin-3-ylamine

A suspension of 4-bromo-6-methoxy-[1,5]naphthyridine-3-carboxylic acid (50 g, 177 mmol) in dry DMF (600 mL) was treated with triethylamine (222.5 mL, 1.60 mole), tert-butanol (265 mL, 2.77 mole) and diphenylphosphoryl azide (41.75 mL, 194 mmol). The reaction was stirred under argon at 100° C. for 1 h, then was cooled to room temperature and concentrated to low volume. Ethyl acetate and excess aqueous sodium bicarbonate solution were added, the mixture was shaken, and some insoluble solid was filtered off. The layers were separated and the organic phase was washed with water (2x) and dried (MgSO₄). Concentration to dryness gave a crude mixture of 4-bromo-6-methoxy-[1,5]naphthyridin-3-ylamine (minor product) and (4-bromo-6-methoxy-[1,5]naphthyridin-3-ylamine)carbamic acid tert-butyl ester (major product) along with impurities.

Without further purification, this mixture was dissolved in CH₂Cl₂ (150 mL) and treated with trifluoroacetic acid (100 mL). The reaction was stirred for 3 h then was concentrated to dryness. The residue was partitioned between CHCl₃ and saturated sodium bicarbonate solution and the layers were separated. The aqueous phase was extracted with CHCl₃, and the combined organic fractions were dried (MgSO₄) and concentrated to low volume. The solid was collected by suction filtration, washed with a small volume of CHCl₃ and dried under vacuum to afford a first crop of the title compound (31.14 g). The filtrate was purified by flash chromatography on silica gel (30% EtOAc in CHCl₃) to afford further material (2.93 g, total=34.07 g, 76%). Alternatively, the filtrate was left at room temperature overnight and then filtered to give a second crop of the title compound (2.5 g).

(f) 4-Bromo-6-methoxy-[1,5]naphthyridine-3-diazonium tetrafluoroborate

A solution of 4-bromo-6-methoxy-[1,5]naphthyridin-3-ylamine (25.2 g, 99.2 mmol) in dry THF (400 mL) was maintained at −5° C. while nitrosonium tetrafluoroborate (12.9 g, 110 mmol) was added portionwise over 30 min (approximately 2 g portions). The reaction was continued for an additional 1 h at −5° C., at which time TLC* and LC/MS indicated that the reaction was complete. The orange solid was collected by suction filtration, washed with ice-cold THF and dried under vacuum to provide the title compound (31.42 g, 90%).

(q) 4-Bromo-3-fluoro-6-methoxy-[1,5]naphthyridine

A suspension of 4-bromo-6-methoxy-[1,5]naphthyridine-3-diazonium tetrafluoroborate (31.42 g, 89.0 mmol) in decalin (mixed isomers, 500 mL) in a 2 L flask* was heated to 180° C. and held at this temperature for 5 min. The mixture was cooled and diluted with CHCl₃ (500 mL, to keep the product in solution), and the resulting mixture was stirred vigorously for 30 min to break up a black solid by-product. The mixture was then poured onto a column of silica gel and the column was eluted with CHCl₃ to remove decalin and then with 3% EtOAc/CHCl₃ to afford the title compound (9.16 g, 40%).

(h) 8-ethenyl-7-fluoro-2-(methyloxy)-1,5-naphthyridine

To a solution of 8-bromo-7-fluoro-2-(methyloxy)-1,5-naphthyridine (2.0 g, 7.81 mmol), potassium carbonate (1.08 g, 7.81 mmole), tetrakis-triphenylphosphine (90 mg, 0.08 mmole) in DME (60 mL) and H₂O (20 mL) was added 2,4,6-trivinylcycloborane-pyridine complex (0.94 g, 3.91 mmole). After stirring for 10 hours at 85° C. the reaction contents were concentrated and the product purified by chromatography on silica gel (hexanes/EtOAc, 4:1) to give a low melting solid (1.43 g, 90%).

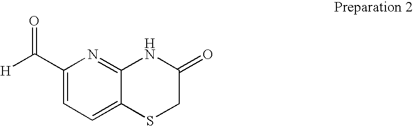

### Preparation of 3-Oxo-3,4-dihydro-2H-pyrido[3,2-b][1,4]thiazine-6-carboxaldehyde

(a) Methyl 3-oxo-3,4-dihydro-2H-pyrido[3,2-b][1,4]thiazine-6-carboxylate

A solution of ethyl 2-mercaptoacetate (1.473 mL) in DMF (48 mL) was ice-cooled and treated with sodium hydride (540 mg of a 60% dispersion in oil). After 1 h methyl 6-amino-5-bromopyridine-2-carboxylate (3 g) (T. R. Kelly and F. Lang, J. Org. Chem. 61, 1996, 4623-4633) was added and the mixture stirred for 16 h at room temperature. The solution was diluted with EtOAc (1 L), washed with water (3×300 mL), dried and evaporated to about 10 mL. The white solid was filtered off and washed with a little EtOAc to give the ester (0.95 g); LC/MS (APCl⁻) m/z 223 ([M-H]⁻, 100%).

(b) 3-Oxo-3,4-dihydro-2H-pyrido[3,2-b][1,4]thiazine-6-carboxvlic acid

A solution of Methyl 3-oxo-3,4-dihydro-2H-pyrido[3,2-b][1,4]thiazine-6-carboxylate (788 mg) in dioxan (120 ml)/water (30 mL) was treated dropwise over 2 h with 0.5 M NaOH solution (8 mL) and stirred overnight. After evaporation to approx. 3 ml, water (5 mL) was added and 2M HCl to pH4. The precipitated solid was filtered off, washed with a small volume of water and dried under vacuum to give a solid (636 mg); LC/MS (APCl⁻) m/z 209 ([M-H]⁻, 5%), 165([M-COOH]⁻, 100%).

(c) 6-Hydroxymethyl-3-oxo-3,4-dihydro-2H-pyrido[3,2-b][1,4]thiazine

A solution of 3-Oxo-3,4-dihydro-2H-pyrido[3,2-b][1,4]thiazine-6-carboxylic acid (500 mg) in THF (24 mL) with triethylamine (0.396 mL) was cooled to −10° C. and isobutyl chloroformate (0.339 ml) was added. After 20 minutes the suspension was filtered through kieselguhr into an ice-cooled solution of sodium borohydride (272 mg) in water (8 mL), the mixture stirred 30 minutes and the pH reduced to 7 with dilute HCl. The solvent was evaporated and the residue triturated under water. The product was filtered and dried under vacuum to give a white solid (346mg); LC/MS (APCl⁻) m/z 195 ([M-H]⁻, 50%), 165 (100%).

(d) 3-Oxo-3,4-dihydro-2H-pyrido[3,2-b][1,4thiazine-6-carboxaldehyde

A solution of 6-Hydroxymethyl-3-oxo-3,4-dihydro-2H-pyrido[3,2-b][1,4]thiazine (330 mg) in dichloromethane (30 mL)/THF (30 mL) was treated with manganese dioxide (730 mg) and stirred at room temperature. Further manganese dioxide was added after 1 h (730 mg) and 16 h (300 mg). After a total of 20 h the mixture was filtered through kieselguhr and the filtrate evaporated. The product was triturated with EtOAc/hexane (1:1) and collected to give a solid (180 mg); LC/MS (APCl⁻) m/z 195 ([M-H]⁻, 95%),165 (100%).

### EXAMPLE 1

Preparation of (±)-6-{[((3aR,4R,6aS)-2-{2-[3-fluoro-6-(methyloxy)-1,5-naphthyridin-4-yl]ethylloctahydrocyclopenta[c]pyrrol-4-yl)amino]methyl}-2H-pyrido[3,2-b][1,4]thiazin-3(4H)-one
(a) 2-(phenylmethyl)octahydrocyclopentarclpyrrol-4-amine

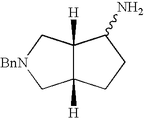

To a solution of 2-(phenylmethyl)hexahydrocyclopenta[c]pyrrol-4(1H)-one (1.4 g, 6.5 mmol) in DCM (2.5 mL) were added hydroxylamine hydrochloride (679 mg, 9.8 mmol), MeOH (25 mL) and 10% NaOH (3.8 mL, 6.5 mmol, 10% in H₂0). After heating at 50° C. for 0.5 h, the resulting solution was cooled, concentrated and partitioned between DCM and H₂O. The aqueous phase was extracted several times with DCM. The organic fractions were combined and concentrated to afford a brown oil. The oil was dissolved in THF (2 mL) and added dropwise to a solution of LAH (0.93 mL, 1M in THF) in THF (3 mL). After refluxing for 0.5 h, the reaction mixture was cooled to 0° C. and subsequently quenched by dropwise addition of a saturated solution of potassium sodium tartrate. The aqueous phase was extracted several times with DCM and the combined organic fractions were dried (Na₂SO₄) and concentrated. The diastereomers were separated using column chromatography (silica, 0-10% MeOH in chloroform (1% NH₄OH)) affording (3aR,4R,6aS)-2-(phenylmethyl)octahydrocyclopenta[c]pyrrol-4-amine (616 mg, 47%) and (3aR,4S,6aS)-2-(phenylmethyl)octahydrocyclopenta[c]pyrrol-4-amine (178 mg, 17%) both as white foams: LCMS (ES) m/e 217 (M+H)⁺.
(b) 1,1-dimethylethyl [(3aR,4R,6aS)-2-(Phenylmethyl)octahydrocyclopentarclpyrrol-4-yl]carbamate

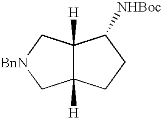

To a solution of (4R)-2-(phenylmethyl)octahydrocyclopenta[c]pyrrol-4-amine (616 mg, 2.85 mmol) in THF (30 mL) at 25° C. was added bis(1,1-dimethylethyl) dicarbonate (746 mg, 3.42 mmol) After 2 h, the solution was concentrated and purified by column chromatography (silica, 2% MeOH in DCM (1% NH₄OH)) providing the title compound as an off-white solid (700 mg, 78%): LC/MS (ES) m/e 317 (M+H)⁺.
(c) 1,1-dimethylethyl (3aR,4R.6aS)-octahydrocyclopentarclpyrrol-4-ylcarbamate

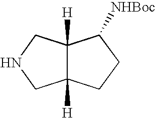

To a solution of 1,1-dimethylethyl [(3aR,4R,6aS)-2-(phenylmethyl)octahydrocyclopenta[c]pyrrol-4-yl]carbamate (700 mg, 2.22 mmol) in EtOH (22 mL) was added Pd(OH)₂ (280 mg, 30 wt %). The suspension was hydrogenated at 50 psi using a Parr shaker. After 5 h, the mixture was filtered through Celite and washed several times with MeOH. The filtrate was concentrated to afford the title compound (330 mg, 66%) as a white solid, which was used without further purification; LC/MS (ES) m/e 227 (M+H)⁺.
(d) 1,1-dimethylethyl ((3aR,4R,6aS)-2-f2-[3-fluoro-6-(methyloxy)-1,5-naphthyridin-4-yl]ethyl}octahydrocyclopenta[c]pyrrol-4-yl)carbamate

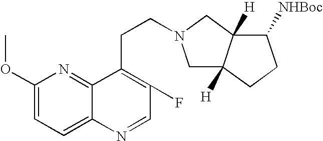

8-ethenyl-7-fluoro-2-(methyloxy)-1,5-naphthyridine (248 mg, 1.22 mmol) and 1,1-dimethylethyl (3aR,4R,6aS)-octahydrocyclopenta[c]pyrrol-4-ylcarbamate (275 mg, 1.22 mmol) in EtOH (3 mL) were heated to 85 ° C. After 12 h, the solution was concentrated and the residue was purified via column chromatography (silica,1% MeOH in DCM (1% NH₄OH)) yielding the title compound as a yellow foam (418 mg, 80%): LC/MS (ES) m/e 431 (M+H)⁺.
(e) (3aR,4R,6aS)-2-f2-r3-fluoro-6-(methyloxy)-1 ,5-naphthyridin-4-yl]ethyl}octahydrocyclopenta[c]pyrrol-4-amine

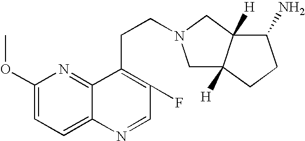

To a solution of 1,1-dimethylethyl ((3aR,4R,6aS)-2-{2-[3-fluoro-6-(methyloxy)-1,5-naphthyridin-4-yl]ethyl}octahydrocyclopenta[c]pyrrol-4-yl)carbamate (418 mg, 0.972 mmol) in MeOH (10 mL) at 25° C. was added dropwise an HCl solution (2 mL, 8.0 mmol, 4M HCl in dioxane). After 12 h, the solution was concentrated to afford an orange residue, which was dissolved in DCM and treated with DIPEA (0.5 mL). The solution was concentrated and washed through a silica pad (5% MeOH in DCM (1% NH₄OH)) to afford the title compound as an orange oil (300 mg, 94%): LC/MS (ES) m/e 320 (M+H)⁺.
(f) (±)-6-{[((3aR,4R,6aS)-2-{2-[3-fluoro-6-(methyloxv)-1,5-naphthyridin-4-yl]ethyl}octahydrogyclopenta[c]pyrrol-4-yl)amino]methyl}-2H-pyrido[3,2-b][1,4]thiazin-3(4M-one

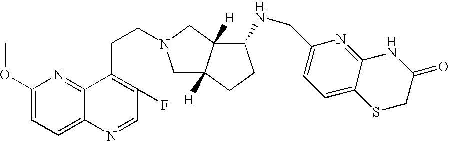

To a solution of (3aR,4R,6aS)-2-{2-[3-fluoro-6-(methyloxy)-1,5-naphthyridin-4-yl]ethyl}octahydrocyclopenta[c]pyrrol-4-amine (150 mg, 0.455 mmol) in DCM:EtOH (5 mL, 1:1) were added Na₂SO₄ (97 mg, 0.68 mmol) and 3-oxo-3,4-dihydro-2H-pyrido [3,2-b][1,4]thiazine-6-carbaldehyde (89 mg, 0.455 mmol). After 12 h at 25° C., NaBH₄ (34 mg, 0.88 mmol) was added. Following an additional 1 h, the reaction was concentrated and the residue was partitioned between DCM-H₂O. The aqueous phase was extracted several times with DCM and the combined organic fractions were dried over MgSO₄, concentrated and purified via column chromatography (silica, 0-1% MeOH in DCM (1% NH₄OH)) yielding the title compound (104 mg, 49%) as an off-white solid: LC/MS (ES) m/e 509 (M+H)⁺; ¹H NMR (CD₃OD, 400 Hz) 67 8.65 (s,1H), 8.21 (d, J=9.0 Hz,1H), 7.70 (d, J=7.8 Hz, 1H), 7.19 (d, J=9.0 Hz, 1H), 7.03 (d, J=7.8 Hz, 1H), 4.11 (s, 3H), 3.79 (s, 2H), 3.52 (s, 2H), 3.47-3.49 (m, 2H), 3.22-3.29 (m, 1H), 3.09-3.17 (m, 1H), 2.91-2.97 (m, 3H), 2.72-2.83 (m, 2H), 2.46-2.50 (m, 1H), 2.13-2.17 (m, 1H), 1.81-1.85 (m, 1H), 1.67-1.73 (m, 1H), 1.5-1.58 (m, 2H).

This material, as a solution in MeOH, was treated with an excess of 4M HCl in dioxane and evaporated to dryness to provide the dihydrochloride salt of the title compound as a yellow solid.

### EXAMPLE 2

Preparation of (±)-6-{[((3aR,4S,6aS)-2-{2-[3-fluoro-6-(methyloxy)-1,5-naphthyridin-4-yl]ethyl}octahydrocyclopenta[c]pyrrol-4-yl)amino]methyl}-2H-pyrido[3,2-b][1,4]thiazin-3(4H)-one

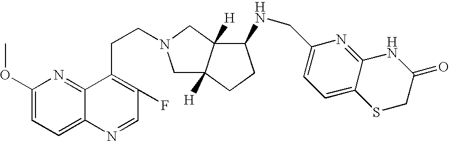

The title compound (35 mg, 18%) was prepared according to Example 1, except substituting (3aR,4S,6aS)-2-(phenylmethyl)octahydrocyclopenta[c]pyrrol-4-amine (120 mg, 0.377 mmol) for (3aR,4R,6aS)-2-(phenylmethyl)octahydrocyclopenta[c]pyrrol-4-amine: LC/MS (ES) m/e 509 (M+H)⁺; ¹H NMR (CD₃OD,400 MHz) δ 8.64 (s,1 H), 8.22 (d, J=9.0 Hz, 1H), 7.71 (d, J=7.8 Hz, 1H), 7.19 (d, J=9.0 Hz, 1H), 7.04 (d, J=7.8 Hz, 1H), 4.12 (s, 3H), 3.83 (s, 2H), 3.51 (s, 2H), 3.46-3.50 (m, 2H), 3.24-3.27 (m, 2H), 2.84-2.91 (m, 2H), 2.62-2.77 (m, 3H), 2.56-2.59 (m, 1H), 2.41-2.49 (m, 1H), 1.92-2.02 (m, 2H), 1.48-1.55 (m, 1H), 1.31-1.42 (m, 1H).

This material, as a solution in MeOH, was treated with an excess of 4M HCl in dioxane and evaporated to dryness to provide the dihydrochloride salt of the title compound.

### EXAMPLE 3

Preparation of 6-{[((3aR,6aS)-2-{2-[3-fluoro-6-(methyloxy)-1,5-naphthyridin-4-yl]ethyl}octahydrocyclopenta[c]pyrrol-5-yl)amino]methyl-2H-pyrido[3,2-b][1,4]thiazin-3(4H)-one
(a) 1,1-dimethylethyl (3aR,6aS)-5-aminohexahydrocyclopenta[c]pyrrole-2(1H)-carboxylate

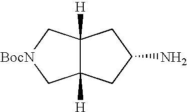

To a solution of 1,1-dimethylethyl 5-oxohexahydrocyclopenta[c]pyrrole-2(1)-carboxylate (145 mg, 0.644 mmol) in MeOH (4 mL) was added ammonium formate (508 mg, 8.05 mmol). After 15 min. at 25° C., the solution was treated with NaCNBH₃ (202 mg, 3.2 mmol). After an additional 12 h, the resulting solution was concentrated and partitioned between DCM and 1 N NaOH. The aqueous phase was extracted several times with DCM and the combined organic fractions were concentrated and purified by column purification ( silica, 1-5% MeOH in DCM (1% NH₄OH)) affording the title compound as a brown oil (110 mg, 76%): LC/MS (ES) m/e 227 (M+H)⁺.
(b) 1,1-dimethylethyl (3aR,6aS)-5-{[(3-oxo-3,4-dihydro-2H-pyrido[3,2-b][1,4]thiazin-6-yl)methyl]amino}hexahydrocyclopenta[c]pyrrole-2(1H)-carboxylate

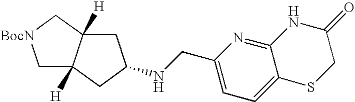

A solution of 1,1-dimethylethyl (3aR,6aS)-5-aminohexahydrocyclopenta[c]pyrrole-2(1H-carboxylate (120 mg, 0.359 mmol), 3-oxo-3,4-dihydro-2H-pyrido[3,2-b][1,4]thiazine-6-carbaldehyde (59 mg, 0.359 mmol) and Na₂SO₄ (60 mg, 0.431 mmol) in DCM-EtOH (4 mL, 1:1) was stirred at 25° C. over 12 h. NaBH₄ (24 mg, 0.431 mmol) was added and the solution stirred an additional 2 h., was concentrated and partitioned between H₂O-DCM. The aqueous phase was washed several times with DCM and the combined organic fractions were dried (Na₂SO₄), concentrated and purified by column chromatography yielding the title compound (92 mg, 46%) as an off-white solid: LC/MS (ES) m/e 405 (M+H)⁺.
(c) 6-{[(3aR,5r,6aS)-octahydrocyclopenta[c]pyrrol-5-ylamino]methyl}-2H-pyrido[3,2-b] [1,4]thiazin-3(4H)-one

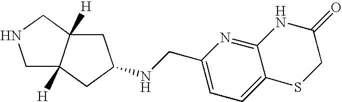

To a solution of 1,1-dimethylethyl (3aR,6aS)-5-{[(3-oxo-3,4-dihydro-2H-pyrido[3,2-b][1,4]thiazin-6-yl)methyl]amino}hexahydrocyclopenta[c]pyrrole-2(1H-carboxylate (92 mg, 0.228 mmol) in MeOH (3 mL) at 25° C. was added dropwise a solution of HCl in dioxane (0.29 mL, 1.14 mmol, 4M HCl in dioxane). After 12 h, the solution was concentrated and the residue dissolved in DCM and neutralized with DIPEA (0.5 mL). The mixture was concentrated and washed through silica (5% MeOH in DCM (1% NH₄OH)) to afford the title compound as an orange oil (70 mg, quant.): LC/MS (ES) m/e 305 (M+H)⁺.
(d) 6-{[((3aR,6aS)-2-{2-[3-fluoro-6-(methyloxy)-1,5-naphthyridin-4-yl]ethyl} octahydrocyclopenta[c]pyrrol-5-yl)amino]methyl}-2H-pyrido[3,2-b] [1,4]thiazin-3(4H)-one

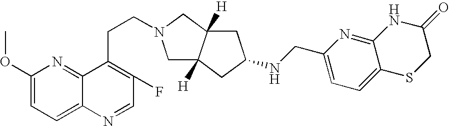

8-ethenyl-7-fluoro-2-(methyloxy)-1,5-naphthyridine (50 mg, 0.231 mmol) and 6-{[(3aR,6aS)-octahydrocyclopenta[c]pyrrol-5-ylamino]methyl}-2H-pyrido[3,2-b][1,4]thiazin-3(4H)-one (70 mg, 0.231 mmol) in EtOH (0.1 mL) were stirred at 85° C. for 12 h. The solution was then concentrated and the residue purified via column chromatography (silica, 1 % MeOH in DCM (1% NH₄OH)) yielding the title compound (25 mg, 21 %): LC/MS (ES) m/e 509 (M+H)⁺; ¹H NMR (CD₃OD,400 MHz) δ 8.52 (s, 1 H), 8.08 (d, J=9.1 Hz, 1H), 7.54 (d, J=7.8 Hz, 1H), 7.05 (d, J=9.1 Hz, 1 H), 6.89 (d, J=7.8 Hz, 1H), 3.98 (s, 3H), 3.62 (s, 2H), 3.37 (s, 2H), 3.28-3.32 (m, 2H), 2.89-2.94 (m, 1 H), 2.73-2.76 (m, 2H), 2.66-2.68 (m, 2H), 2.40-2.52 (m, 4H), 2.05-2.10 (m, 2H), 1.18-1.26 (m, 2H).

This material, as a solution in MeOH, was treated with an excess of 4M HCl in dioxane and evaporated to dryness to provide the dihydrochloride salt of the title compound.

### EXAMPLE 4

Preparation of (±)-6-{[(3aR,6aS)-5-{2-[3-fluoro-6-(methyloxy)-1,5-naphthyridin-4-yl]ethyl}hexahydropyrrolo[3,4-c]pyrrol-2(1 H)-yl]methyl}-2H-pyrido[3,2-b][1,4]thiazin-3(4H)-one

(a) 2,5-bis(phenylmethyl)tetrahydropyrrolo[3,4-c]pyrrole-1,3(2H,3aH)-dione

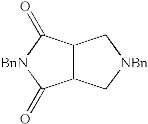

To a solution of [(methyloxy)methyl](phenylmethyl)[(trimethylsilyl)methyl]amine (1.27 g, 5.34 mmol) and 1-(phenylmethyl)-1H-pyrrole-2,5-dione (0.5 g, 2.67 mmol) in DCM (13 mL) at 0° C. was added TFA (21 μL, 0.267 mmol). The solution warmed to 25° C. over 3 h and was partitioned between sat. NaHCO₃ and DCM. The aqueous phase was extracted several times with DCM. The combined organic fractions were dried (Na₂SO₄), concentrated and purified by column chromatography (silica, 0.5% MeOH in chloroform) to afford the title compound as an off-white solid (600 mg, 70%): LC-MS m/z 321 (M+H)⁺.
(b) 5-(phenylmethyl)tetrahydropyrrolo[3,4-c]pyrrole-1,3(2H,3aH)-dione

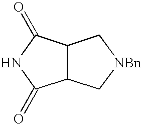

To a solution of 2,5-bis(phenylmethyl)tetrahydropyrrolo[3,4-c]pyrrole-1,3(2H,3aH-dione (730 mg, 2.28 mmol) in EtOH (22 mL) was added 10% Pd/C (220 mg, 30 wt %). The suspension was hydrogenated using a hydrogen balloon at 25° C. After 12 h, the mixture was filtered and washed several times with MeOH. The filtrate was concentrated and purified by column chromatography (silica, 2% MeOH in DCM (1% NH₄OH)) to afford the title compound (367 mg, 70%) as an off-white solid: LC/MS (ES) m/e 231 (M+H)⁺.
(c) 2-(phenylmethyl)octahydropyrrolo[3,4-c]pyrrole

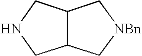

To a solution of 5-(phenylmethyl)tetrahydropyrrolo[3,4-c]pyrrole-1,3(2H,3aH)-dione (367 mg, 1.59 mmol) in THF (16 mL) at 0° C. was added dropwise a solution of LAH (7.2 mL, 7.2 mmol, 1M in THF). The reaction warmed to 25° C. and was heated to 40° C. for an additional 1h. Upon cooling to 0° C., the reaction was quenched by dropwise addition of a saturated solution of potassium sodium tartrate. The aqueous phase was extracted several times with DCM and the combined organic fractions were dried over Na₂SO₄, concentrated and purified by column chromatography (silica, 10% MeOH in DCM (1% NH₄OH)) yielding the title compound as a yellow oil (212 mg, 66%): LCMS (ES) mle 203 (M+H)⁺.
(d) 7-fluoro-2-(methyloxy)-8-{2-[(3aR,6aS)-5-(phenylmethyl)hexahydropyrrolo[3,4-c]pyrrol-2(1H)-yl]ethyl}-1,5-naphthyridine

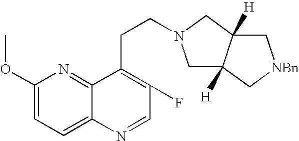

A solution of 8-ethenyl-7-fluoro-2-(methyloxy)-1,5-naphthyridine (214 mg, 1.05 mmol) and 2-(phenylmethyl)octahydropyrrolo[3,4-c]pyrrole (212 mg, 1.05 mmol) in EtOH (1 mL) was heated for 12 h at 80° C. The solution was concentrated and the residue purified via column chromatography (silica, 1% MeOH in DCM (1% NH4OH)) yielding the title compound (260 mg, 61 %) as a yellow foam: LC/MS (ES) m/e 407 (M+H)⁺.
(e) 7-fluoro-8-{2-[(3aR,6aS)-hexahydropyrrolo[3,4-c]pyrrol-2(1H)-yl]ethyl}-2-(methyloxy)-1,5-naphthyridine

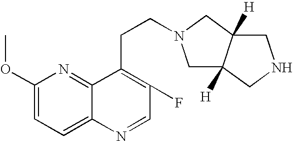

To a solution of 7-fluoro-2-(methyloxy)-8-{2-[(3aR,6aS)-5-(phenylmethyl)hexahydropyrrolo[3,4-c]pyrrol-2(1H)-yl]ethyl}-1,5-naphthyridine (260 mg, 0.64 mmol) in EtOH (6 mL) was added Pd(OH)₂ (78 mg, 30 wt %). The suspension was hydrogenated at 50 psi using a Parr shaker. After 12 h, the mixture was filtered through Celite® and washed several times with MeOH. The filtrate was concentrated to afford the title compound as a yellow oil (172 mg, 85%) which was used without further purification: LC/MS (ES) m/e 317 (M+H)⁺.
(f)(±)-6-{[(3aR,6aS)-5-{2-[3-fluoro-6-(methyloxy)-1,5-naphthyridin-4-yl]ethyl}hexahydropyrrolo[3,4-c]pyrrol-2(l1 H-yl]methyl}-2H-pyrido[3,2-b][1,4]thiazin-3(4M-one

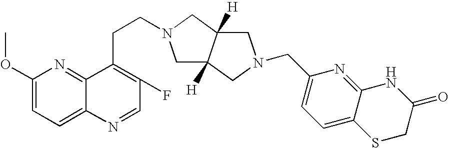

To a solution of 7-fluoro-8-{2-[(3aR,6aS)-hexahydropyrrolo[3,4-c]pyrrol-2(1 H)-yl]ethyl}-2-(methyloxy)-1,5-naphthyridine (177 mg, 0.56 mmol) in DCM (6 mL) at 25° C. were added 3-oxo-3,4-dihydro-2H-pyrido[3,2-b][1,4]thiazine-6-carbaldehyde (109 mg, 0.56 mmol) followed by NaBH(OAc)₃ (178 mg, 0.84 mmol). After 1 h, the reaction was concentrated and the residue was partitioned between DCM and a saturated aqueous solution of NaHCO₃. The aqueous phase was extracted several times with DCM and the combined organic fractions were dried over MgSO₄, concentrated and purified via column chromatography (silica, 0-1.5% MeOH in DCM (1 % NH₄OH)) yielding the title compound (178 mg, 64%) as a yellow solid: LC/MS (ES) m/e 495 (M+H)⁺; ¹H NMR (CD₃OD, 400 Hz) δ 8.67 (s, 1H), 8.23 (d, J=9.0 Hz, 1H), 7.70 (d, J =7.8 Hz, 1H), 7.20 (d, J=9.0 Hz, 1H), 7.07 (d, J=7.8 Hz, 1H), 4.13 (s, 3H), 3.62 (s, 2H), 3.53 (s, 2H), 3.47-3.49 (m, 2H), 2.76-2.90 (m, 8H), 2.54-2.57 (m, 2H), 2.39-2.41 (m, 2H).

This material, as a solution in MeOH, was treated with an excess of 4M HCl in dioxane and evaporated to dryness to provide the dihydrochloride salt of the title compound as a yellow solid.

### EXAMPLE 5

Preparation of (±)-6-[({[(3aS,6aR)-2-{2-[3-fluoro-6-(methyloxy)-1,5-naphthyridin-4-yl]ethyl}hexahydrocyclopenta[c]pyrrol-3a(l1 -yl]methyl}amino)methyl]-2H-pyrido[3,2-b][1,4]thiazin-3(4H)-one
(a) methyl (3aR)-2-(phenylmethyl)hexahydrocyclopenta[c]pyrrole-3a(1H)-carboxylate

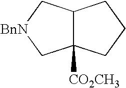

The title compound (620 mg, 60%) was prepared as a colorless solid according to Example 4, except substituting methyl 1-cyclopentene-1-carboxylate (500 mg, 3.96 mmol) for 1-(phenylmethyl)-1H-pyrrole-2,5-dione: LC-MS m/z 260 (M+H)⁺.
(b) [(3aR)-2-(phenylmethyl)hexahydrocyclopenta[c]pyrrol-3a(1H)-yl]methanol

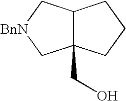

To a solution of methyl (3aR)-2-(phenylmethyl)hexahydrocyclopenta[c]pyrrole-3a(1H-carboxylate (620 mg, 2.39 mmol) in THF (24 mL) at 0° C. was added dropwise a solution of LAH (5.2 mL, 5.2 mmol, 1M in THF). After 0.5 h at 0° C., the mixture was quenched by dropwise addition of a saturated solution of potassium sodium tartrate. The aqueous phase was extracted several times with DCM and the combined organic fractions were dried over Na₂SO₄, concentrated and purified by column chromatography (silica, 5% MeOH in DCM (1% NH₄OH)) yielding the title compound as a yellow oil (500 mg, 90%):

LCMS (ES) m/e 231 (M+H)⁺.
(c)[(3aR,6aR)-2-(2-[3-fluoro-6-(methyloxy)-1,5-naphthyridin-4-yl]ethyl}hexahydrocyclopenta[c]pyrrol-3a(1H)-yl]methanol

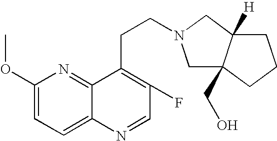

A solution of 8-ethenyl-7-fluoro-2-(methyloxy)-1,5-naphthyridine (475 mg, 2.33 mmol) and [(3aR)-2-(phenylmethyl)hexahydrocyclopenta[c]pyrrol-3a(1H)-yl]methanol (328 mg, 2.33 mmol) in EtOH (1 mL) was heated to 85° C. After 12 h, the solution was concentrated and the residue purified via column chromatography (silica, 1% MeOH in DCM (1% NH4OH)) yielding the title compound (530 mg, 93%) as a clear oil: LC/MS (ES) m/e 246 (M+H)⁺.
(d) 2-{[(3aR,6aR)-2-{2-[3-fluoro-6-(methyloxy)-1,5-naphthyridin-4-yl]ethyl}hexahydrocyclopenta[c]pyrrol-3a(1 H-yl]methyl}-1H-isoindole-1,3(2H)-dione

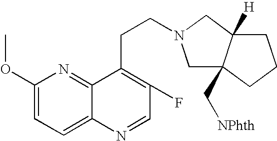

A solution of [(3aR,6aR)-2-{2-[3-fluoro-6-(methyloxy)-1,5-naphthyridin-4-yl]ethyl}hexahydrocyclopenta[c]pyrrol-3a(1H)-yl]methanol (280 mg, 0.81 mmol) and DEAD (0.14 mL, 0.89 mmol) in THF (2 mL) at 25° C. was treated with a solution of phthalimide (119 mg, 0.81 mmol) and PPh₃ (213 mg, 0.81 mmol) in THF-dioxane (3 mL, 2:1). After 12 h at 70° C., the solution was concentrated and the residue purified by column chromatography (silica, 0.5% MeOH in DCM (1% NH₄OH)) providing the title compound as a yellow foam (384 mg, quant.): LC/MS (ES) m/e 475 (M+H)⁺.
(e) {[(3aS,6aR)-2-{2-[3-fluoro-6-(methyloxy)-1,5-naphthyridin-4-yl]ethyl}hexahydrocyclopenta[c]pyrrol-3a(1 H)-yl]methyl}amine

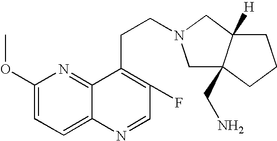

To a solution of 2-{[(3aR,6aR)-2-{2-[3-fluoro-6-(methyloxy)-1,5-naphthyridin-4-yl]ethyl}hexahydrocyclopenta[c]pyrrol-3a(1 H)-yl]methyl}-1H-isoindole-1,3(2H)-dione (385 mg, 0.812 mmol) in EtOH (41 mL) was added NH₂NH₂ hydrate (0.379 mL, 12.2 mmol). After stirring at reflux for 2 h, the solution was concentrated and purified by column chromatography (silica, 3% MeOH in DCM (1 % NH₄OH)) to afford the title compound (160 mg, 57%): LC/MS (ES) m/e 345 (M+H)⁺.
(f) (±)-6-[({[(3aS,6aR)-2-{2-[3-fluoro-6-(methyloxy)-1,5-naphthyridin-4-yl]ethyl}hexahydrocyclopenta[c]pyrrol-3a(1H)-yl]methyl}amino)methyl]-2H-pyrido [3,2-b][1,4]thiazin-3(4M-one

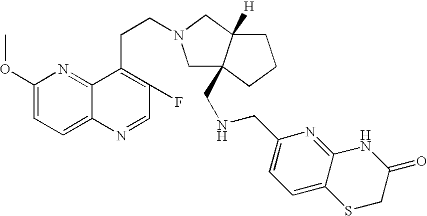

To a solution of {[(3aS,6aR)-2-{2-[3-fluoro-6-(methyloxy)-1,5-naphthyridin-4-yl]ethyl}hexahydrocyclopenta[c]pyrrol-3a(1 h)-yl]methyl}amine (160 mg, 0.465 mmol) in DCM/EtOH (7.5 mL, 2:1) were added Na₂SO₄ (99 mg, 0.698 mmol) and 3-oxo-3,4-dihydro-2H-pyrido[3,2-b][1,4]thiazine-6-carbaldehyde (91 mg, 0.47 mmol). After 12 h at 25° C., NaBH₄ (26 mg, 0.69 mmol) was added. After an additional 1 h, the reaction was concentrated and the residue was partitioned between DCM and H₂O. The aqueous phase was extracted several times with DCM and the combined organic fractions were dried over MgSO₄, concentrated and purified via column chromatography (silica, 0.5-1% MeOH in DCM (1% NH₄OH)) yielding the title compound (130 mg, 54%) as a yellow solid: LC/MS (ES) m/e 522 (M+H)⁺; ¹H NMR (CD₃OD, 400 Hz) δ 8.63 (s, 1H), 8.19 (d, J=9.0 Hz, 1H), 7.67 (d, J=7.8Hz, 1H), 7.17 (d, J=9.0Hz, 1H), 6.99 (d, J=7.8Hz, 1H), 4.11 (s, 3H), 3.76 (s, 2H), 3.52 (s, 2H), 3.41-3.45 (m, 2H), 2.99-23.08 (m, 2H), 2.69-2.85 (m, 2H), 2.64 (d, J=11.2 Hz, 1H), 2.54 (d, J=11.3 Hz, 1H), 2.25-2.28 (m, 1H), 2.17-2.19 (m, 2H), 1.57-1.73 (m, 5H), 1.45-1.49 (m,1H).

This material, as a solution in MeOH, was treated with an excess of 4M HCl in dioxane and evaporated to dryness to provide the dihydrochloride salt of the title compound as a yellow solid.

| Example | Structure | Formula |
|---|---|---|
| 1 | 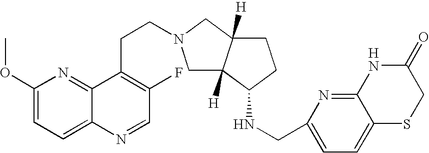 | 6-{[((3aS,4S,6aR)-2-{2-[3-fluoro-6- (methyloxy)-1,5-naphthyridin-4- yl]ethyl}octahydrocyclopenta[c]pyrrol-4- yl)amino]methyl}-2H-pyrido[3,2- b][1,4]thiazin-3(4H)-one |
| 2 | 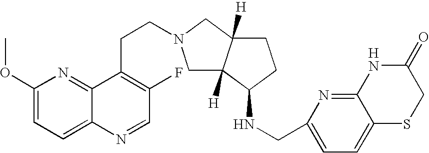 | 6-{[((3aS,4R,6aR)-2-{2-[3-fluoro-6- (methyloxy)-1,5-naphthyridin-4- yl]ethyl}octahydrocyclopenta[c]pyrrol-4- yl)amino]methyl}-2H-pyrido[3,2- b][1,4]thiazin-3(4H)-one |
| 3 | 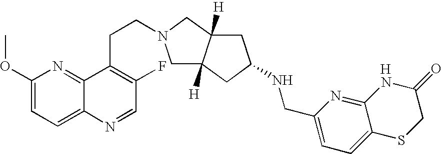 | 6-{[((3aR,6aS)-2-{2-[3-fluoro-6- (methyloxy)-1,5-naphthyridin-4- yl]ethyl}octahydrocyclopenta[c]pyrrol-5- yl)amino]methyl}-2H-pyrido[3,2- b][1,4]thiazin-3(4H)-one |
| 4 | 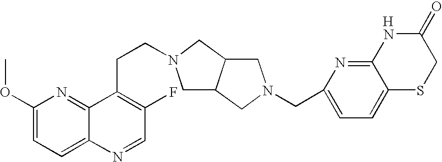 | 6-{[5-{[3-fluoro-6-(methyloxy)-1,5- naphthyridin-4- yl]ethyl}hexahydropyrrolo[3,4-c]pyrrol- 2(1H)-yl]methyl}-2H-pyrido[3,2-b][1,4]thiazin-3(4H)-one |
| 5 | 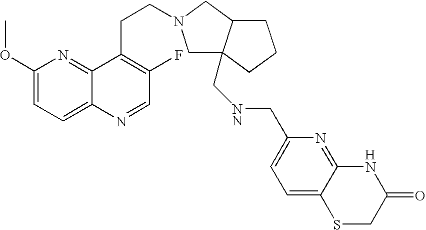 | 6-[({[2-{2-[3-fluoro-6-(methyloxy)-1,5- naphthyridin-4- yl]ethyl}hexahydrocyclopenta[c]pyrrol- 3a(1H)-yl]methyl}amino)methyl]-2H- pyrido[3,2-b][1,4]thiazin-3(4H)-one |

### EXAMPLE 6

Antimicrobial Activity Assay:

Whole-cell antimicrobial activity was determined by broth microdilution using the National Committee for Clinical Laboratory Standards (NCCLS) recommended procedure, Document M7-A6, “Methods for Dilution Susceptibility Tests for Bacteria that Grow Aerobically”. The compounds were tested in serial two-fold dilutions ranging from 0.016 to 16 mcg/mL.

Compounds were evaluated against a panel of Gram-positive organisms, including Staphylococcus aureus, Streptococcus pneumoniae, Streptococcus pyogenes, Enterococcus faecalis and Enterococcus faecium.

In addition, compounds were evaluated against a panel of Gram-negative strains including Haemophilus influenzae, Moraxella catarrhalis, Escherichia coli, Pseudomonas aeruginosa, Proteus mirabilis, Enterobacter cloacae, Enterobacter aerogenes, Klebsiella pneumoniae and Stenotrophomonas maltophilia.

The minimum inhibitory concentration (MIC) was determined as the lowest concentration of compound that inhibited visible growth. A mirror reader was used to assist in determining the MIC endpoint.

One skilled in the art would consider any compound with a MIC of less than 20 mg/mL to be a potential lead compound. For instance, each of the listed Examples (1 to 5), as identified in the present application, had a MIC≦20 mg/ml against at least one of the organisms listed above.

It is to be understood that the invention is not limited to the embodiments illustrated hereinabove and the right is reserved to the illustrated embodiments and all modifications coming within the scope of the following claims.

1. A compound of formula (I) 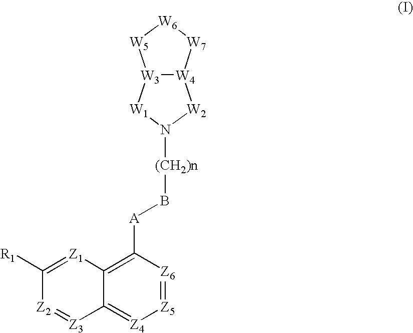
wherein:
Z₁, Z₃, and Z₄ are independently N or CR^{1a};
Z₂, Z₅, and Z₆ are each CR^{1a};
R₁ and R^{1a} are independently at each occurrence hydrogen; cyano; halogen; hydroxy; (C₁₋₆)alkoxy unsubstituted or substituted by (C₁₋₆)alkoxy, hydroxy, amino, piperidyl, guanidino or amidino any of which is unsubstitued or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C₁₋₆)alkyl, acyl or (C₁₋₆)alkylsulphonyl groups; or R₁ and R^{1a} of Z₂ together form ethylenedioxy;
AB is NR^{1b}(C=O); NR^{1b}; C(=O)CR₂R₃; or CR₂R₃CR₄R₅;
R^{1b} and R^{1b} are independently at each occurrence hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heteroaryl; heteroaralkyl; or heterocyclyl;
R₂, R₃, R₄, R₅ and R₆ are independently at each occurrence hydrogen; thiol; (C₁₋₆)alkylthio; halogen; trifluoromethyl; azido; (C1-6)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl or aralkyl;
R^{1c} and R^{1c′} are independently at each occurrence hydrogen; (C₁₋₆)alkyl; aralkyl; aryl; heteroarylalkyl; heteroaryl; heterocyclyl; or together with the nitrogen that they are attached form an aziridine, azetidine, pyrrolidine, piperidine or hexamethyleneimine ring (wherein said aziridine, azetidine, pyrrolidine, piperidine or hexamethyleneimine ring are optionally substiuted with from 1 to 3 substituents selected from halogen, hydroxy; cyano; nitro; (C₁₋₆)alkyl; and aryl);
n, n′ and n″ are independently and at each occurrence integers from 0 to 2;
W₁ and W₂ are each CR₆R₇;
R₇ is independently at each occurrence hydrogen, (C₁₋₆)alkyl; aryl; or heteroaryl;
W₃ and W₄ are each CR₈;
R₈ is independently at each occurrence hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; aralkyl; or R₉;
R₉ is UR^{1d};
U is (CH₂)ₙNR^{1b}(CH₂)_{n′}; (CH₂)ₙNR^{1b}S(O)_{n′}(CH₂)_{n″}; (CH₂)ₙNR^{1b}(C=O)(CH₂)_{n′}; (CH₂)ₙNR^{1b}C(=O)NR^{1b′}(CH₂)_{n′}; (CH₂)ₙNR^{1b}(CO₂)(CH₂)_{n′}; (CH₂)ₙS(CH₂)_{n′}; or (CH2)ₙO(CH2)ₙ;
W₅, W₆ and W₇ are independently CR₁₀R₁₁ or NR₁₂;
R₁₀ is independently at each occurrence hydrogen; acyloxy; thiol; (C₁₋₆)alkylthio; halogen; trifluoromethyl; azido; (C1-6)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; or aralkyl;
R₁₁ is independently at each occurrence hydrogen; (C₁₋₆)alkyl; aryl; heteroaryl; or R₉;
R₁₂ is independently at each occurrence hydrogen, trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heteroaryl; heteroaralkyl; heterocyclyl; or R₁₃;
R₁₃ is U′R^{1d};
U′ is (CH₂)ₙ or (C=O)(CR₂R₃)ₙ;
R^{1d} is a substituted or unsubstituted bicyclic carbocyclic or heterocyclic ring system (A): 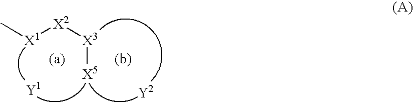
containing up to four heteroatoms in each ring in which at least one of rings (a) and (b) is aromatic;
X¹ is C or N when part of an aromatic ring or CR₁₄ when part of a non aromatic ring;
X² is N, NR₁₅, O, S(O)ₙ, CO or CR₁₄ when part of an aromatic or non-aromatic ring or may in addition be CR₁₆R₁₇ when part of a non aromatic ring;
X³ and X⁵ are independently N or C;
Y¹ is a 0 to 4 atom linker group each atom of which is independently selected from N, NR₁₅, O, S(O)ₙ, CO and CR₁₄ when part of an aromatic or non-aromatic ring or may additionally be CR₁₆R₁₇ when part of a non aromatic ring,
Y² is a 2 to 6 atom linker group, each atom of Y² being independently selected from N, NR₁₅, O, S(O)ₙ, CO and CR₁₄ when part of an aromatic or non-aromatic ring or may additionally be CR₁₆R₁₇ when part of a non aromatic ring;
R₁₄, R₁₆and R₁₇ are at each occurrence independently selected from: H; (C₁₋₄)alkylthio; halo; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;
R₁₅ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted with (C₁₋₄)alkyl;
or a pharmaceutically acceptable salt thereof;
provided that the compound of formula (I) contains one R₉ or R₁₃ substituent.

2. A compound or salt according to claim 1, wherein Z₁ and Z₄ are N and Z₃ is CR^{1a}.

3. A compound or salt according to claim 1, wherein R₁ is OCH₃.

4. A compound or salt according to claim 1, wherein R^{1a} is at each occurrence independently hydrogen; halogen; or cyano.

5. A compound or salt according to claim 1, wherein AB is CR₂R₃CR₄R₅.

6. A compound or salt according to claim 5, wherein R₂, R₃, R4 and R₅ are each hydrogen.

7. A compound or salt according to claim 1, wherein:
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅ and W₇ are each CR₁₀R₁₁;
R₁₀ is hydrogen; hydroxy; (C₁₋₆)alkyl; acyloxy; or halogen;
R₁₁ is hydrogen; (C₁₋₆)alkyl; aryl; or heteroaryl;
W₆ is NR₁₂; and
R₁₂ is R₁₃.

8. A compound or salt according to claim 1, wherein:
R₆ is independently at each occurrence hydrogen; hydroxy; halogen; or (C₁₋₆)alkyl;
R₇ is independently at each occurrence hydrogen or (C₁₋₆)alkyl;
R₈ of W₃ is hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted by hydrogen, (C₁₋₆)alkyl, (C₂₋₆)alkenyl; or aralkyl;
R₈ of W₄ is R₉;
W₅, W₆ and W₇ are each CR₁₀R₁₁; and
R₁₁ is hydrogen; (C₁₋₆)alkyl; aryl; or heteroaryl.

9. A compound or salt according to claim 1, wherein:
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅, W₆ and W₇ are each CR₁₀R₁₁;
R₁₀ is hydrogen; hydroxy; (C₁₋₆)alkyl; acyloxy; or halogen;
R₁₁ of W₅ and W₇ is independently at each occurrence hydrogen; (C₁₋₆)alkyl; aryl; or heteroaryl; and
R₁₁ of W₆ is R₉.

10. A compound or salt according to claim 1, wherein:
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅, W₆ and W₇ are each CR₁₀R₁₁;
R₁₀ is hydrogen; hydroxy; (C₁₋₆)alkyl; acyloxy; or halogen;
R₁₁ of W₅ and W₆ is independently at each occurrence hydrogen; (C₁₋₆)alkyl; aryl; or heteroaryl; and
R₁₁ is W7 is R₉.

11. A compound or salt according to claim 1, wherein R^{1d} is:
4H-Pyrido[3,2-b][1,4]thiazin-3-oxo-6-yl;
4H-Pyrido[3,2-b]oxazin-3-oxo-6-yl;
2,3-Dihydro-benzo[1,4]dioxin-6-yl;
4H-benzo[1,4]thiazin-3-oxo-6-yl;
2,3-Dihydro-furo[2,3-c]pyridin-5-yl;
4H-Pyrido[3,2-b]oxazin-3-oxo-6-yl;
7-Chloro-4H-pyrido[3,2-b]oxazin-3-oxo-6-yl;
2,3-Dihydro-[1,4]dioxino[2,3-c]-pyridin-6-yl;
2,3-Dihydro-benzofuran-7-carbonitrile-5-yl;
7-Methyl-4H-pyrido[3,2-b][1,4]thiazin-3-oxo-6-yl;
3-Oxa-1-thia-5-aza-indan-5-yl;
5-Methyl-2,3-dihydro-benzo[1,4]dioxin-6-yl;
6-Fluoro-2,3-dihydro[1,4]dioxin-7-yl;
2,3-Dihydro-benzofuran-5-yl;
7-Fluoro-4H-benzo[1,4]thiazin-3-oxo-6-yl;
4H-Benzo[1,4]thiazin-3-oxo-6-yl; or
8-Methyl-2,3-dihydro-benzo[1,4]dioxin-6-yl.

12. A compound or salt according to claim 1, wherein:
Z₁ and Z₄ are N;
Z₃ is CR^{1a};
R₁ is OCH₃;
R^{1a} is at each occurrence independently hydrogen; halogen; or cyano;
AB is CH₂CH₂;
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅, W₆ and W₇ are each CR₁₀R₁₁;
R₁₀ is independently at each occurrence hydrogen; hydroxy; (C₁₋₆)alkyl; acyloxy; or halogen;
R₁₁ of W₅ and W6 is independently at each occurrence hydrogen; (C₁₋₆)alkyl; aryl; or heteroaryl; and
R₁₁ of W₇ is R₉.

13. A compound or salt according to claim 12, wherein:
U is (CH₂)ₙNR^{1b}(CH₂)_{n′};
R^{1b} is hydrogen or (C₁₋₆)alkyl; and
R^{1d} is 4H-Pyrido[3,2-b][1,4]thiazin-3-oxo-6-yl;
4H-Pyrido[3,2-b]oxazin-3-oxo-6-yl;
2,3-Dihydro-benzo[1,4]dioxin-6-yl;
4H-benzo[1,4]thiazin-3-oxo-6-yl;
2,3-Dihydro-furo[2,3-c]pyridin-5-yl;
4H-Pyrido[3,2-b]oxazin-3-oxo-6-yl;
7-Chloro-4H-pyrido[3,2-b]oxazin-3-oxo-6-yl;
2,3-Dihydro-[1,4]dioxino[2,3-c]-pyridin-6-yl;
2,3-Dihydro-benzofuran-7-carbonitrile-5-yl;
7-Methyl-4H-pyrido[3,2-b][1,4]thiazin-3-oxo-6-yl;
3-Oxa-1-thia-5-aza-indan-5-yl;
5-Methyl-2,3-dihydro-benzo[1,4]dioxin-6-yl;
6-Fluoro-2,3-dihydro[1,4]dioxin-7-yl;
2,3-Dihydro-benzofuran-5-yl;
7-Fluoro-4H-benzo[1,4]thiazin-3-oxo-6-yl;
4H-Benzo[1,4]thiazin-3-oxo-6-yl; or
8-Methyl-2,3-dihydro-benzo[1,4]dioxin-6-yl.

14. A compound or salt according to claim 1, wherein:
Z₁ and Z₄ are N;
Z₃ is CR^{1a};
R₁ is OCH₃;
R^{1a} is at each occurrence independently hydrogen; halogen; or cyano;
AB is CH₂CH₂;
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅, W₆ and W₇ are each CR₁₀R₁₁;
R₁₀ is independently at each occurrence hydrogen; hydroxy; (C₁₋₆)alkyl; acyloxy; or halogen;
R₁₁ of W₅ and W₇ is independently at each occurrence hydrogen; (C₁₋₆)alkyl; aryl; or heteroaryl; and
R₁₁ of W₈ is R₉.

15. A compound or salt according to claim 14, wherein:
U is (CH₂)ₙNR^{1b}(CH₂)_{n′};
R^{1b} is hydrogen or (C₁₋₆)alkyl; and
R^{1d} is 4H-Pyrido[3,2-b][1,4]thiazin-3-oxo-6-yl;
4H-Pyrido[3,2-b]oxazin-3-oxo-6-yl;
2,3-Dihydro-benzo[1,4]dioxin-6-yl;
4H-benzo[1,4]thiazin-3-oxo-6-yl;
2,3-Dihydro-furo[2,3-c]pyridin-5-yl;
4H-Pyrido[3,2-b]oxazin-3-oxo-6-yl;
7-Chloro-4H-pyrido[3,2-b]oxazin-3-oxo-6-yl;
2,3-Dihydro-[1,4]dioxino[2,3-c]-pyridin-6-yl;
2,3-Dihydro-benzofuran-7-carbonitrile-5-yl;
7-Methyl-4H-pyrido[3,2-b][1,4]thiazin-3-oxo-6-yl;
3-Oxa-1-thia-5-aza-indan-5-yl;
5-Methyl-2,3-dihydro-benzo[1,4]dioxin-6-yl;
6-Fluoro-2,3-dihydro[1,4]dioxin-7-yl;
2,3-Dihydro-benzofuran-5-yl;
7-Fluoro-4H-benzo[1,4]thiazin-3-oxo-6-yl;
4H-Benzo[1,4]thiazin-3-oxo-6-yl; or
8-Methyl-2,3-dihydro-benzo[1,4]dioxin-6-yl.

16. A compound or salt according to claim 1; wherein
Z₁ and Z₄ are N;
Z₃ is CR^{1a};
R₁ is OCH₃;
R^{1a} is at each occurrence independently hydrogen; halogen; or cyano;
AB is CH₂CH₂;
R₆ is independently at each occurrence hydrogen; hydroxy; halogen; or (C₁₋₆)alkyl;
R₇ is independently at each occurrence hydrogen or (C₁₋₆)alkyl;
R₈ of W₃ is hydrogen; (C₁₋₆)alkyl; hydroxy; or halogen;
R₈ of W₄is R₉;
W₅, W₆ and W₇ are each CR₁₀R₁₁;
R₁₀ is independently at each occurrence hydrogen; hydroxy; (C₁₋₆)alkyl; acyloxy; or halogen; and
R₁₁ is independently at each occurrence hydrogen; (C₁₋₆)alkyl; aryl; or heteroaryl.

17. A compound or salt according to claim 16, wherein:
U is (CH₂)ₙNR^{1b}(CH₂)_{n′};
R^{1b} is hydrogen or (C₁₋₆)alkyl; and
R^{1d} is 4H-Pyrido[3,2-b][1,4]thiazin-3-oxo-6-yl;
4H-Pyrido[3,2-b]oxazin-3-oxo-6-yl;
2,3-Dihydro-benzo[1,4]dioxin-6-yl;
4H-benzo[1,4]thiazin-3-oxo-6-yl;
2,3-Dihydro-furo[2,3-c]pyridin-5-yl;
4H-Pyrido[3,2-b]oxazin-3-oxo-6-yl;
7-Chloro-4H-pyrido[3,2-b]oxazin-3-oxo-6-yl;
2,3-Dihydro-[1,4]dioxino[2,3-c]-pyridin-6-yl;
2,3-Dihydro-benzofuran-7-carbonitrile-5-yl;
7-Methyl-4H-pyrido[3,2-b][1,4]thiazin-3-oxo-6-yl;
3-Oxa-1 -thia-5-aza-indan-5-yl;
5-Methyl-2,3-dihydro-benzo[1,4]dioxin-6-yl;
6-Fluoro-2,3-dihydro[1,4]dioxin-7-yl;
2,3-Dihydro-benzofuran-5-yl;
7-Fluoro-4H-benzo[1,4]thiazin-3-oxo-6-yl;
4H-Benzo[1,4]thiazin-3-oxo-6-yl; or
8-Methyl-2,3-dihydro-benzo[1,4]dioxin-6-yl.

18. A compound or salt according to claim 1, wherein:
Z₁ and Z₄ are N;
Z₃ is CR^{1a};
R₁ is OCH₃;
R^{1a} is at each occurrence independently hydrogen; halogen; or cyano;
AB is CH₂CH₂;
R₆ is independently at each occurrence hydrogen; hydroxy; halogen; or (C₁₋₆)alkyl;
R₇ is independently at each occurrence hydrogen or (C₁₋₆)alkyl;
R₈ is independently at each occurrence hydrogen; (C₁₋₆)alkyl; hydroxy; or halogen;
W₅ and W₇ are each CR₁₀R₁₁;
R₁₀ is independently selected from hydrogen; hydroxy; (C₁₋₆)alkyl; acyloxy; or halogen;
R₁₁ is independently at each occurrence hydrogen; (C₁₋₆)alkyl; aryl; or heteroaryl;
W₆ is NR₁₂; and
R₁₂ is R₁₃.

19. A compound or salt according to claim 18, wherein
U′ is (CH₂)ₙ; and
R^{1d} is 4H-Pyrido[3,2-b][1,4]thiazin-3-oxo-6-yl;
4H-Pyrido[3,2-b]oxazin-3-oxo-6-yl;
2,3-Dihydro-benzo[1,4]dioxin-6-yl;
4H-benzo[1,4]thiazin-3-oxo-6-yl;
2,3-Dihydro-furo[2,3-c]pyridin-5-yl;
4H-Pyrido[3,2-b]oxazin-3-oxo-6-yl;
7-Chloro-4H-pyrido[3,2-b]oxazin-3-oxo-6-yl;
2,3-Dihydro-[1,4]dioxino[2,3-c]-pyridin-6-yl;
2,3-Dihydro-benzofuran-7-carbonitrile-5-yl;
7-Methyl-4H-pyrido[3,2-b][1,4]thiazin-3-oxo-6-yl;
3-Oxa-1-thia-5-aza-indan-5-yl;
5-Methyl-2,3-dihydro-benzo[1,4]dioxin-6-yl;
6-Fluoro-2,3-dihydro[1,4]dioxin-7-yl;
2,3-Dihydro-benzofuran-5-yl;
7-Fluoro-4H-benzo[1,4]thiazin-3-oxo-6-yl;
4H-Benzo[1,4]thiazin-3-oxo-6-yl; or
8-Methyl-2,3-dihydro-benzo[1,4]dioxin-6-yl.

20. A compound according to claim 1, wherein the compound is:
a) (±)-6-{[((3aR,4R,6aS)-2-{2-[3-fluoro-6-(methyloxy)-1,5-naphthyridin-4-yl]ethyl}octahydrocyclopenta[c]pyrrol-4-yl)amino]methyl}-2H-pyrido[3,2-b][1 ,4]thiazin-3(4H)-one;
b) (±)-6-{[((3aR,4S,6aS)-2-{2-[3-fluoro-6-(methyloxy)-1,5-naphthyridin-4-yl]ethyl}octahydrocyclopenta[c]pyrrol-4-yl)amino]methyl}-2H-pyrido[3,2-b][1 ,4]thiazin-3(4H)-one;
c) 6-{[((3aR,6aS)-2-{2-[3-fluoro-6-(methyloxy)-1,5-naphthyridin-4-yl]ethyl}octahydrocyclopenta[c]pyrrol-5-yl)amino]methyl}-2H-pyrido[3,2-b][1 ,4]thiazin-3(4H)-one;
d) (±)-6-{[(3aR,6aS)-5-{2-[3-fluoro-6-(methyloxy)-1,5-naphthyridin-4-yl]ethyl}hexahydropyrrolo[3,4-c]pyrrol-2(1H)-yl]methyl}-2H-pyrido[3,2-b][1,4]thiazin-3(4H)-one; or
e) (±)-6-[({[(3aS,6aR)-2-{2-[3-fluoro-6-(methyloxy)-1,5-naphthyridin-4-yl]ethyl}hexahydrocyclopenta[c]pyrrol-3a( 1 H)-yl]methyl}amino)methyl]-2H-pyrido[3,2-b][1,4]thiazin-3(4H)-one; or
a pharmaceutically acceptable salt thereof.

21-22. (canceled)

23. A pharmaceutical composition comprising a compound or salt according to claim 1 and a pharmaceutically acceptable carrier.

24. A method of treating bacterial infections in mammals which comprises administering to a mammal in need thereof an effective amount of a compound or salt according to claim 1.
